(19) United States
(12) Patent Application Publication
(10) Pub. No.: US 2007/0072239 A1
(43) Pub. Date: Mar. 29, 2007

(54) Pharmaceutical compositions and methods of using secreted frizzled related protein

(75) Inventors: Ramesh A. Bhat, King of Prussia, PA (US); Peter Van Nest Bodine, Havertown, PA (US)

Correspondence Address: DARBY & DARBY P.C., P.O. BOX 5257, NEW YORK, NY, 10150-5257, US

(73) Assignee: Wyeth, Madison, NJ (US)

(21) Appl. No.: 11/515,501

(22) Filed: Sep. 1, 2006

Related U.S. Application Data
(60) Provisional application No. 60/722,890, filed on Sep. 30, 2005.
(60) Provisional application No. 60/720,952, filed on Sep. 26, 2005.

(51) Int. Cl. G01N 33/53; C07H 21/04; C12P 21/06; C07K 14/705; C12N 5/08

(52) U.S. Cl. 435/7.1; 435/69.1; 435/320.1; 435/366; 530/350; 536/23.5

(57) ABSTRACT

Methods and pharmaceutical compositions of use in regulation of mammalian bone-forming activities of SFRPs (secreted frizzled-related proteins) are disclosed. SFRPs are secreted receptors for Wnts, which are important polypeptide growth factors that are known to regulate fundamental biological processes such as tissue polarity, embryonic development, and tumorigenesis. Methods can be used to identify compounds which target the Wnt-antagonizing function of an SFRP by way of one or more domains or specific amino acids. Pharmaceutical compositions can be prepared using these compounds, and bone disorders can be treated using these pharmaceutical compositions.

### CROSS-REFERENCE TO RELATED APPLICATIONS

Priority is claimed under 35 U.S.C. § 119(e) to U.S. Provisional Application Ser. No. 60/722,890, filed on Sep. 30, 2005 and to U.S. Provisional Application Ser. No. 60/720,952, filed on Sep. 26, 2005. The contents of these priority applications are incorporated into the present disclosure by reference and in their entireties.

### FIELD OF THE INVENTION

The present invention relates to methods that can be used to identify compounds which target the Wnt-antagonizing function of a secreted frizzled-related protein (SFRP) by way of one or more domains or specific amino acids. The present invention also relates to pharmaceutical compositions that can be prepared using these compounds. The pharmaceutical compositions of the present invention can be used to treat bone disorders.

### BACKGROUND OF THE INVENTION

The topic of bone formation regulation and bone-related disorders has gained considerable attention. For example, in the women's health area there has been a particular focus on the bone-related disorder osteoporosis. Throughout life, there is a constant remodeling of skeletal bone. Bone is formed and maintained by two cell types: osteoblasts that synthesize and mineralize the bone matrix, and osteoclasts that resorb the calcified tissue (Komm and Bodine (2001) in Osteoporosis. Marcus et al. eds. Acadermic Pres: San Diego, pages 305-337; Bodine and Komm (2002) Vitam. Horm. 64:101-151; Goltzman (2002) Nat. Rev. Drug Discov. 1:784-796). Osteoblasts arise from multipotent mesenchymal stem cells that are located in bone marrow (Lian et al. (1999) in Primer on the metabolic bone diseases and disorders of mineral metabolism. M. J. Favus, ed. Lippincott Williams & Wilkins: Philadelphia, pages 14-29; Bodine and Komm, (2002) Vitam. Horm. 64:101-151; Goltzman (2002) Nat. Rev. Drug Discov. 1:784-796), while osteoclasts originate from hematopoietic bone marrow cells (Teitelbaum (2000) Science 289:1504-1508; Goltzman (2002) Nat. Rev. Drug Discov. 1:784-796). Osteoblast and osteoclast cells work together in a process known as bone remodeling, which is the mechanism by which immature, damaged, or aged bone is replaced with new lamellar bone (Mundy (1999) in Primer on the metabolic bone diseases and disorders of mineral metabolism. Favus, ed. Lippincott Williams & Wilkins: Philadelphia, pages 30-38). Bone remodeling is initiated by recruitment and activation of osteoclasts that remove the mineralized matrix. The process ends about 6 months later with the filling-in of the resorption pit with newly formed osteoid by the osteoblasts. At the end of this last phase, the bone-forming cells experience one of three fates (Manolagas (2000) Endocr. Rev. 21:115-137; Bodine and Komm, (2002) Vitam. Horn. 64:101-151; Goltzman (2002) Nat. Rev. Drug Discov. 1:784-796). They can differentiate to osteocytes upon entrapment within the mineralized matrix; they can differentiate to quiescent lining cells; or they can undergo apoptosis.

The majority of studies on age-related changes in human bone have been directed toward elucidating changes in bone on a morphological level or by quantitatively comparing rates of bone loss. Disruption of the fine balance between the differentiation of new osteoclast and osteoblast cells and the timing of cell death by apoptosis is thought to be an important mechanism behind bone loss disorders. Therapeutic agents that alter the prevalence of apoptosis in osteoblasts and/or osteoclasts are useful and desirable to correct the imbalance in cell numbers that is the basis of diminished bone mass and increased risk of fractures in osteoporosis (for review, see, Manolagas (2000) Endocr. Rev. 21:115-137; Weinstein and Manolagas (2000) Am. J. Med. 108:153-164).

### The Wnt Gene Family

One group of genes and the proteins encoded by them that play an important role in regulating cellular development is the Wnt family of glycosylated lipoproteins. Wnt proteins are a family of growth factors consisting of more than a dozen structurally related molecules that are involved in the regulation of fundamental biological processes such as apoptosis, embryogenesis, organogenesis, morphogenesis, and tumorigenesis (reviewed in Nusse and Varmus (1992) Cell 69:1073-1087). These polypeptides are multipotent factors and have similar biological activities to other secretory proteins such as transforming growth factor (TGF)-β, fibroblast growth factors (FGFs), nerve growth factor (NGF), and bone morphogenetic proteins (BMPs). Members of the Wnt family related to bone include Wnt3 (human sequence set forth in SEQ ID NO: 27), Wnt1 (human sequence set forth in SEQ ID NO: 26), and Wnt10b. Wnt10b endogenously regulates bone formation by increasing bone mass and bone strength, conferring resistance to the loss of bone associated with aging, protecting against bone loss due to ovariectomy, and stimulating osteoblastogenesis (Bennett et al. (2005) Proc. Natl. Acad. Sci. USA 102:3324-3329).

### The Frizzled Family of Proteins

Studies indicate that certain Wnt proteins interact with a family of proteins named “Frizzled” (or “Fz,” “Fzd,” or “FZD”) that act as receptors for Wnt proteins or as components of a Wnt receptor complex (reviewed in Moon et al. (1997) Cell 88:725-728; Barth et al. (1997) Curr. Opin. Cell Biol. 9:683-690). Frizzled proteins contain an amino terminal signal sequence for secretion, a cysteine-rich domain (CRD) that is thought to bind Wnt, seven putative transmembrane domains that resemble a G-protein coupled receptor, and a cytoplasmic carboxyl terminus.

The discovery of the first secreted frizzled-related protein (SFRP) was reported by Hoang et al. ((1996) J. Biol. Chem. 271:26131-26137). This protein, which was called “Frzb” for frizzled motif in bone development, was purified and cloned from bovine articular cartilage extracts based on its ability to stimulate in vivo chondrogenic activity in rats. The human homologue of the bovine gene was also cloned. However, unlike the frizzled proteins, Frzb did not contain a serpentine transmembrane domain. Thus, this new member of the frizzled family appeared to be a secreted receptor for Wnt. The Frzb cDNA encoded for a 325 amino acid/36,000 Dalton (Da) protein that was predominantly expressed in the appendicular skeleton. The highest level of expression was in developing long bones and corresponded to epiphyseal chondroblasts; expression then declined and disappeared toward the ossification center.

The SFRP family of proteins are ˜32-40 kiloDalton (kDa) glycoproteins that were identified as antagonists of Wnt signaling (Rattner et al. (1997) Proc. Natl. Acad. Sci. USA 94:2859-63; Melkonyan et al. (1997) Proc. Natl. Acad. Sci. USA 94:13636-41; Finch et al. (1997) Proc. Natl. Acad. Sci. USA 94:6770-5; Uren et al. (2000) J. Biol. Chem. 275:4374-82; Kawano et al. (2003) J. Cell. Sci. 116:2627-34). In mammals, there are five SFRPs, grouped into two subfamilies based on sequence homology. SFRP-1 (human sequence set forth in SEQ ID NO: 4) is most closely related to SFRP-5 (human sequence set forth in SEQ ID NO: 8) and SFRP-2 (human sequence set forth in SEQ ID NO: 5) (56% and 36% amino acid similarity respectively) and is more distantly related to SFRP-3 (human sequence set forth in SEQ ID NO: 6) and SFRP-4 (human sequence set forth in SEQ ID NO: 7) (19% and 17% amino acid similarity respectively; see FIG. 8 for an alignment of these proteins). The SFRPs contain three structural units: an amino terminal signal peptide, a Frizzled (FZD) type cysteine-rich domain (CRD), and a carboxy-terminal netrin domain. The CRD spans ˜120 amino acids, contains 10 conserved cysteine residues and has 30-50% sequence similarity to the CRD of FZD receptors. The disulphide linkage and the cysteine spacing of human SFRP-1 (SEQ ID NO: 4) has been determined, and the cysteine spacing of the CRD is highly conserved throughout the homologs and orthologs (Chong et al. (2002) J. Biol. Chem. 277:5134-44). Crystallographic data of the CRD of mouse SFRP-3 and mFZD 8 have been resolved, and the structures have revealed the potential for the CRD to homodimerize or heterodimerize between SFRP and FZD (Dann et al. (2001) Nature 412:86-90). Several biochemical studies have shown the interaction of the SFRP CRD and Wnt and also the complex formation of the CRDs of FZD and SFRPs (Bafico et al. (1999) J. Biol. Chem. 274:16180-16187). Such findings suggest that SFRP inhibition of Wnt signaling may operate through at least two mechanisms: (i) by competition with FZD for Wnt ligands, or (ii) in a dominant-negative fashion by direct formation of non-signaling inactive complexes with FZD receptors (Bafico et al. (1999) J. Biol. Chem. 274:16180-16187; Jones et al. (2002) BioEssays 24:811-820).

The carboxyl-terminal half of SFRPs contains a domain that shares some sequence similarity with the axon guidance protein, netrin (Serafini et al. (1994) Cell 78:409-24). This netrin domain is defined by six cysteine residues and several conserved segments of hydrophobic residues and secondary structures. Such a structural domain has also been found in tissue inhibitors of metalloproteinases, Typel procollagen C-proteinase enhancer proteins, and complement proteins C3, C4, and C5 (Banyai et al. (1999) Protein Sci. 8:1636-42). The netrin domain in SFRP-1 and SFRP-5 contains a highly charged hyaluronan-binding domain that is responsible for the heparin-binding properties of the protein (Uren et al. (2000) J. Biol. Chem. 275:4374-82). The hyaluronan binding region is shown to be involved in the interaction of SFRP-1 with Wingless, the Drosophila ortholog of mammalian Wnt-1 (Uren et al. (2000) J. Biol. Chem. 275:4374-82).

The biological activity of SFRPs is largely attributed to their role as regulators of Wnt function. Several studies have suggested a role in the regulation of apoptosis (Melkonyan et al. (1997) Proc. Natl. Acad. Sci. USA 94:13636-41; Chong et al. (2002) J. Biol. Chem. 277:5134-44; Han et al. (2004) J. Biol. Chem. 279:2832-2840). In a knockout mouse model, deletion of mouse SFRP-1 led to decreased osteoblast and osteocyte apoptosis, increased osteoprogenitor differentiation, enhanced bone formation and elevated bone mineral density (Bodine et al. (2004) Mol. Endocrinol. 18:1222-37). Thus, some SFRPs have been identified as “SARPs” for secreted apoptosis related proteins. The five known human SFRP/SARP genes are SFRP-1/FrzA/FRP-1/SARP-2, SFRP-2/SDF-5/SARP-1, SFRP-3/Frzb-1/FrzB/Fritz, SFRP-4 and SFRP-5/SARP-3 (Leimeister et al. (1998) Mech. Dev. 75:29-42).

Using a phage display library, a peptide motif that bound to SFRP-1 has been identified (L/V-VDGRW-L/W; SEQ ID NO: 29) (Chuman et al. (2004) Peptides 25:1831-8) and the interaction of SFRP-1 with RANKL that contained the peptide motif has been demonstrated. Such an interaction of SFRP-1 and RANKL led to the inhibition of osteoclast formation (Hausler et al. (2004) J. Bone Miner. Res. 19:1873-81). Thus the biological role of SFRP-1 has expanded into new avenues beyond its role as a regulator of Wnt action.

### SFRPs and Bone Formation

SFRP-1 and the Wnt signaling pathway have been found to be involved in the regulation of bone formation (Westendorf et al. (2004) Gene 341:19-29). Inhibition of SFRP-1 promotes increased rate of bone formation, a decrease in osteoblast and osteoclast apoptosis. and an increase in osteoblast differentiation. (For review, see, Bodine et al. (2004) Mol. Endocrinol. 18:1222-37.) A need exists for the definitive identification of targets for the treatment of bone disorders (including bone formation disorders and bone density disorders) and degenerative bone disorders, including osteodegeneration disorders (osteopenia, osteoarthritis, and osteoporosis).

There exists a need in the art to better understand the critical domains and amino acids of the SFRP family of proteins. Once identified, these domains. and amino acids can be targeted to regulate Wnt activity and thus bone formation. The present invention fulfills this need by demonstrating which domains and amino acids are important and by providing methods to identify test compounds that bind to SFRPs or change the interaction of SFRPs with FZDs and Wnts.

### SUMMARY OF THE INVENTION

The present invention provides for methods of screening for a test compound that targets in a secreted frizzled related protein (SFRP) one or more of a domain selected from the group consisting of the cysteine rich domain (CRD), the netrin domain, and the hyaluronan domain. The present invention also provides for methods of screening for a test compound that targets in a SFRP one or more of an amino acid selected from the group consisting of the amino acid corresponding to tyrosine 73 of SEQ ID NO: 2, and the amino acids corresponding to lysine 228, 229, 230, 231, 234, 239, 240, 241, 244, and 245 of SEQ ID NO: 2. If the SFRP is SFRP-1, the one or more of an amino acids may be the amino acids corresponding to 73-86 of SEQ ID NO: 2, or the amino acid corresponding to asparagine 173 of SEQ ID NO: 2, or the amino acids corresponding to 296-314 of SEQ ID NO: 2

These methods can comprise several steps, including incubating a first sample comprising a wild-type SFRP and the test compound; incubating a second sample comprising a SFRP identical to the wild-type SFRP, except the SFRP has a mutation within or a deletion of one or more of a domain or amino acid selected from the group described above, and the test compound; and determining the amount of test compound bound to SFRP in each sample, wherein a test compound, that shows increased binding to wild-type SFRP in the first sample compared to the mutant SFRP of the second sample, targets one or more of a domain or amino acid selected from the group described above.

Furthermore, the present invention contemplates a method for identifying a test compound that targets the Wnt-antagonizing function of a SFRP by way of one or more of a domain or amino acid selected from the group described above, comprising incubating a first sample comprising a wild-type SFRP, the test compound and Wnt; incubating a second sample comprising a SFRP identical to the wild-type SFRP, except the SFRP has a mutation within or deletion of a domain or amino acids selected from the group described above, the test compound and Wnt; and determining the Wnt activity in the first and second samples, wherein an increase in Wnt activity in the second sample compared to the first sample indicates the test compound targets the Wnt-antagonizing function of SFRP by way of a domain or amino acids selected from the group described above.

The present invention also contemplates a method of screening for a test compound that targets in a SFRP one or more of a domain selected from the group consisting of a domain or amino acids selected from the group described above, which method comprises incubating a first sample comprising a wild-type SFRP, the test compound and Wnt; incubating a second sample comprising a SFRP identical to the wild-type SFRP, except the SFRP has a mutation at one or more of a domain selected from the group consisting of a domain or amino acids selected from the group described above, the test compound and Wnt; and determining the amount of SFRP bound to Wnt in each sample, wherein a test compound, that decreases the binding of wild-type SFRP and Wnt of the first sample compared to the binding of the mutant SFRP and Wnt of the second sample, targets one or more of a domain or amino acids selected from the group described above.

Furthermore, in addition to using two samples (wild-type SFRP in the presence of a test compound and a mutant SFRP in the presence of the test compound), another embodiment of the invention includes assays such that the amount of binding of SFRP to Wnt, or the activity of Wnt signaling, may be compared to samples of the wild-type SFRP and/or the mutant SFRP in the absence of the test compound. This comparison may provide additional information found useful in determining compounds that target the SFRP.

The present invention also provides for an isolated SFRP, and a nucleic acid encoding an isolated SFRP, in which one or more of a domain or amino acids selected from the group described above, is mutated. In another embodiment, the nucleic acid hybridizes to a nucleic acid that encodes an isolated SFRP. The SFRP may contain a purification tag.

The methods of the present invention, in certain embodiments, may be used where the SFRP is SFRP-1; has a purification tag, including, but not limited to, at the N-terminus, or upstream of the CRD; or has a His purification tag. In other embodiments, the Wnt activity is measured by a T-cell factor luciferase assay. In yet other embodiments, the Wnt is Wnt1, Wnt3, or Wnt10b.

The present invention also provides for small molecules identified by the methods of the invention. Furthermore, pharmaceutical compositions are contemplated comprising a small molecule that targets in a SFRP a domain selected from the group consisting of a domain or amino acids selected from the group described above. In other embodiments, Wnt activity is measured by a T-cell factor luciferase assay; and the Wnt is Wnt1, Wnt3, or Wnt10b. In other embodiments, the pharmaceutical composition is used to regulate bone growth or density.

### BRIEF DESCRIPTION OF THE DRAWINGS

FIG. 1 demonstrates that the CRD domain is critical for Wnt antagonist function of SFRP-1. SFRP-1 (SEQ ID NO: 2) and its CRD mutants were cotransfected along with 16×TCF-Luciferase reporter, CMV-β-gal, and Wnt3 plasmids into U2OS cells. After 20 hrs, the luciferase and β-gal activities in the cell lysates were measured and the results were normalized with β-gal. FIG. 1A shows Wnt activity in the presence of SFRP-1 and its CRD mutants. FIG. 1B shows schematic representations of the sequences on the left with the percent inhibition of Wnt activity on the right.

FIG. 2A is an alignment of SFRP-1 and SFRP-3 CRD sequences (portions of SEQ ID NOS: 4 and 6, respectively). The portion of the CRD that is replaced or mutated is shown in brackets with numbers corresponding to SFRP-1. The tyrosine (amino acid 73) in SFRP-1 is shown by an arrow. Identical amino acids are shown in bold letters; the homologous amino acids are shown in dark shaded rectangles. FIG. 2B Shows the SFRP-1 CRD mutants schematically with the corresponding percent inhibition of Wnt on the right. The SFRP-1 CRD sequence is replaced by the SFRP-3 amino acids in the mutants: The original sequence of SFRP-1 targeted for replacement is shown on the top with amino acid numbers. For the mutants, the amino acid residues in the bold, upper case lettering correspond to the SFRP-3 sequence, and the amino acids in lower case represent the original SFRP-1 amino acids on either side of the SFRP-3 sequence.

FIGS. 3A and 3B are graphs that show a tyrosine residue in the 2^{nd} loop of CRD is critical for Wnt antagonist function of SFRP-1. SFRP-1 and SFRP-3 mutants were assayed for their Wnt inhibitory function. FIG. 3A shows percent Wnt activity. FIG. 3B shows schematic representations of the sequences on the left with percent Wnt inhibition on the right. SFRP-1-660-5 and SFRP-3 show the original tested sequences; bold letters show the mutated amino acids of the SFRP-1 and SFRP-3 mutants; and lower case letters show the original SFRP-1 and SFRP-3 amino acids for the respective mutants.

FIGS. 4A and 4B demonstrate that the netrin domain of SFRP-1 is critical for optimal Wnt antagonist function. FIG. 4A shows an alignment of SFRP-1 and SFRP-2 amino acid sequences corresponding to the netrin domain (portions of SEQ ID NOS: 4 and 5, respectively). Bold letters indicate conserved amino acids. The dark shaded rectangles correspond to the homologous sequences in SFRP-1 and SFRP-2. The arrow represents the crossover in SFRP-1/SFRP-2 chimera. FIG. 4B shows Wnt inhibitory activity of SFRP-1, SFRP-2 and their chimeras. The Wnt3 inhibitory activities are presented as percent Wnt3 activity. FIG. 4C diagrammatically shows the constructs, where the original sequences for SFRP-1 and SFRP-2 are represented in the first two constructs, respectively.

FIG. 5 shows that the hyaluronan binding region in SFRP- 1 is required for its optimal Wnt antagonist function. On the left are schematic representations of the SFRP-1 and its hyaluronan-binding domain mutants. The SFRP-1 region targeted for mutation is shown on the top. Bold letters represent the mutated amino acids. The Wnt3 inhibitory activity is shown on the right as percent Wnt3 inhibition.

FIG. 6 shows Wnt antagonist function of SFRP-1 and SFRP-3 chimeras. On the left are the chimeras drawn schematically, where the original sequences for SFRP-1 and SFRP-2 are represented in the first two constructs, respectively. On the right is the percent inhibition of Wnt.

FIG. 7 shows the effect of glycosylation and position of a His tag on Wnt inhibitory function of SFRP-1. On the left are the schematic diagrams of the sequences with the percent inhibition of Wnt on the right.

FIG. 8 is an alignment of the amino acids encoded by the open reading frames (ORFs) of human SFRP-1 (SEQ ID NO: 4), SFRP-2 (SEQ ID NO: 5), SFRP-3 (SEQ ID NO: 6), SFRP-4 (SEQ ID NO: 7), SFRP-5 (SEQ ID NO: 8).

FIG. 9 shows an alignment of the CRD of SFRP-1 (portion of SEQ ID NO: 4) with several Frizzled CRDs (Fzd 1-10; SEQ ID NOS: 47-56) around the tyrosine 73 of SFRP-1.

### DETAILED DESCRIPTION

SFRP-1 of the present invention (SEQ ID NO: 2) is regulated by osteogenic or bone-forming agents in human osteoblast (hOB) cell lines in vitro. The expression of this gene is upregulated during hOB differentiation, suggesting it may be involved in the bone formation process. DNA sequence analysis indicated that this gene fragment (SEQ ID NO: 1) shared significant sequence identity to a mouse cDNA called secreted frizzled-related protein (SFRP)-1 (Rattner et al. (1997) Proc. Natl. Acad. Sci. USA 94:2859-2863). Subsequent cDNA cloning and additional sequence analysis indicated that the gene of the present invention, which is referred to as the hOB SFRP (SEQ ID NO: 2), was, except for a one amino acid difference at position 174, identical to human SFRP-1/FRP-1/SARP-2 (SEQ ID NO: 4) (U.S. patent application Ser. No. 10/169,545, incorporated herein by reference in its entirety; Finch et al. (1997) Proc. Natl. Acad. Sci. USA 94:6770-6775; Melkonyan et al. (1997) Proc. Natl. Acad. Sci. USA 94:13636-13641). The Wnt antagonist activity of hOB SFRP-1 was found to have no significant difference to the published human SFRP-1.

Development of an SFRP-1 −/− mouse line provided an experimental approach to address the contribution of Wnt signaling to bone biology and to determine if SFRP-1 regulates osteoblast and osteocyte viability in vivo (see US Patent Application Pub. No. 2004/0115195, U.S. Ser. No. 10/666,851, incorporated herein by reference in its entirety). These mice show that deletion of SFRP-1 not only reduces osteoblast and osteocyte apoptosis, but also potentiates osteoprogenitor cell differentiation and increases trabecular bone formation. Targeted deletion of SFRP-1 delays the onset of age-dependent trabecular bone loss, while having little effect on fertility, body weight, blood and urine chemistries, non-skeletal organs or cortical bone. These results indicate that SFRP-1 not only plays a role in the attainment of peak bone mass, but also regulates senile bone loss.

As further disclosed in US Patent Application Pub. No. 2004/0115195 (U.S. Ser. No. 10/666,851), Wnt prolongs the life of human osteoblasts in vitro and antagonism of Wnt signaling by SFRP-1 (SEQ ID NO: 2) promotes cell death. Also, deletion of SFRP-1 in mice results in increased trabecular bone formation, decreased osteoblast and osteocyte apoptosis, enhanced osteoprogenitor differentiation, and enhanced bone marrow-derived osteoprogenitor cell and calvarial-derived osteoblast proliferation without altering bone resorption or skeletal development. Thus, an inhibitor of SFRP-1 function may increase osteoblast/pre-osteocyte survival and therefore enhance bone formation in vivo.

Accordingly, the hOB SFRP-1 (SEQ ID NO: 2) protein may be used as a target and screen for identifying anabolic bone agents and other agents involved in bone formation, repair, and remodeling. Nucleic acids specific for SFRP-1 may be used to inhibit expression of the protein.

Wnt signaling is initiated by the binding of Wnt to a membrane receptor complex composed of the FZD receptor and low-density lipoprotein receptor-related protein (LRP), leading to the activation of the canonical, Wnt/β-catenin, pathway. The activation of Wnt signaling can be measured either by the increase in the cytoplasmic accumulation of P-catenin or the activation of T-cell factor/lymphoid enhancer factor (TCF/LEF)-reporter genes (Wodarz et al. (1998) Annu. Rev. Cell Dev. Biol. 14:59-88; Miller (2002) Genome Biol. 3: reviews 3001.1-3001.15). In such assays, SFRP-1 is shown to decrease the Wnt-mediated accumulation of cytoplasmic β-catenin and inhibit the activation of the TCF-reporter. Microinjection of mRNA into Xenopus embryos is generally used to validate Wnt signaling; and in such a system, Wnt1-induced axis duplication is inhibited by SFRPs (Lin et al. (1997) Proc. Natl. Acad. Sci. USA 94:11196-11200). Biochemical studies utilizing the co-immunoprecipitation or ELISA methods are also used to identify the interaction of Wnt and SFRPs; however; the results of such physical interaction studies do not correlate with in vivo functional studies using Xenopus embryo axis duplication (Lin et al. (1997) Proc. Natl. Acad. Sci. USA 94:11196-11200).

Structure-function studies using bovine SFRP-3 mutants have revealed that the complete removal of the CRD abolishes the Wnt1/SFRP-3 interaction in vitro and the inhibition of the Wnt1-mediated axis duplication in Xenopus embryos (Lin et al. (1997) Proc. Natl. Acad. Sci. USA 94:11196-11200). In contrast, removal of the carboxyl-terminal portion of the molecule preserves both the Wnt-SFRP-3 interaction and reduced functional inhibition of axis duplication. However, studies utilizing human SFRP-1 (SEQ ID NO: 4) and Wg (wingless, a Drosophila Wnt) have shown that the SFRP mutants lacking the CRD retained the ability to bind to Wg, and the deletion of the carboxyl terminal resulted in the reduction or loss of Wg binding. These studies have concluded that the CRD might confer a component of the binding capacity, but the carboxyl-terminal region of the SFRP-1 is primarily responsible for its ability to bind Wg (Uren et al. (2000) J. Biol. Chem. 275:4374-4382). Although the above methods provided the insight into the potential mechanism of the Wnt antagonism of SFRP-1, the studies failed to identify the critical regions that are essential to the biological function of SFRPs.

In the Examples below, an optimized TCF-Luciferase reporter-based assay was used for measuring the Wnt signaling and Wnt antagonist function of SFRPs. A luciferase-based reporter plasmid containing 16 copies of the TCF-element upstream of a tk promoter was developed (Bhat et al. (2004) Protein Expr. Purif. 37:327-335). Several cell lines were analyzed for the optimal Wnt response, and the U2OS cells reproducibly showed a good Wnt response with a nearly 30-fold activation when co-transfected with a Wnt3 expression plasmid. The amount of Wnt and hOB SFRP-1 transfected were optimized to obtain nearly 90% inhibition with SFRP-1. This optimized transfection method allowed characterization of the SFRP-1 mutants and to identify the critical regions that are required for Wnt antagonist function. Using this assay system, it was determined that human SFRP-3 is less efficient in inhibiting Wnt3 compared to SFRP-1, which could be due to the differences in the CRD sequences. A change of the sequences in the 2^{nd} loop of-SFRP-1 compared to those of SFRP-3 have identified that the amino acids between 73-86 play an important role in the Wnt antagonist function of SFRP-1. In particular, the change from KKMVL (SEQ ID NO: 31) to NMTKM (SEQ ID NO: 32) leads to a substantial loss of the antagonist activity. The results correlate very well with studies of alanine scanning mutants of mouse SFRP-3 CRD and its subsequent binding to an XWnt8-AP chimera. Mutations around NMTKM (SEQ ID NO: 32) lead to either reduced or total loss of the binding of the SFRP-3 mutants to XWnt-AP (Dann et al. (2001) Nature 412:86-90). Similarly, a change from LLEHE (SEQ ID NO: 35) to HLHHS (as in SFRP-3; SEQ ID NO: 36) affected the Wnt antagonist function of SFRP-1. In SFRP-3, the H-HHS residues are exposed residues based on fractional solvent solubility studies, and it is possible that subtle changes in the amino acids of SFRPs may alter its secondary and tertiary structures, affecting the Wnt antagonist function of SFRP.

It is intriguing to note the critical role of tyrosine (amino acid 73) in SFRP-1 (human sequence set forth in SEQ ID NO: 4) and its Wnt antagonist function. This tyrosine is conserved in closely related SFRPs like SFRP-1 (human sequence set forth in SEQ ID NO: 4), SFRP-2 (human sequence set forth in SEQ ID NO: 5) and SFRP-5 (human sequence set forth in SEQ ID NO: 8) and is replaced by tryptophan in SFRP-3 (human sequence set forth in SEQ ID NO: 6) and SFRP-4 (human sequence set forth in SEQ ID NO: 7). The tyrosine is also conserved in all of the frizzled receptors. The change of tyrosine to tryptophan in SFRP-1 (human sequence set forth in SEQ ID NO: 4) did not affect its Wnt antagonist function, whereas a change to phenylalanine did result in about a 20% loss of Wnt antagonist function. A more drastic effect on Wnt antagonist function is seen when the aromatic amino acid, tyrosine, is changed into a neutral or polar amino acid such as alanine, serine, and aspartic or asparagine. In crystal structure studies with the mouse SFRP-3 CRD and mFZD8 CRD, the tryptophan/tyrosine residue is buried within the CRD structure. In many proteins, tyrosine residues are generally involved in H bonding, either with other amino acid side chains or with water molecules. The change of the aromatic amino acid tyrosine into a neutral or polar amino acid may disrupt such bonding, altering the folding of the molecule and resulting in the loss of Wnt antagonist function of SFRPs. Preliminary studies with the frizzled receptor have shown that the tyrosine residue in the 2^{nd} loop indeed is critical for the activation of canonical signaling by Wnt ligand. The change of tryptophan to tyrosine in SFRP-3 (human sequence set forth in SEQ ID NO: 6) results in gain of Wnt antagonist activity, suggesting that the tyrosine residue is the favored amino acid residue for optimal Wnt antagonist function of SFRPs.

In the Examples below, the chimera of SFRP-1/SFRP-2 showed reduced Wnt antagonist function, suggesting that the 3′ netrin domain is important for optimal Wnt3 inhibitory activity. In particular, the chimera with the last 19 amino acids of SFRP-1 replaced by 11 amino acids of SFRP-2 showed about 20% of SFRP-1 activity, clearly demonstrating the role of the 3′ sequences in the overall structure of SFRP-1. In contrast, the replacement of the SFRP-2 sequence with SFRP-1 did not result in a significant change in its Wnt3 antagonist activity. These results suggest that the amino acid residues interacting with the carboxyl-terminal region may be different, and a low amino acid homology between SFRP-1 and SFRP-2 (36% similarity) supports that notion.

The SFRP-1 3′ region contains multiple lysine residues, and this hyaluronan binding region is shown to be involved in the binding of SFRP-1 to heparin or heparin sulphate proteoglycan (HSPG) (Uren et al. (2000) J. Biol. Chem. 275:4374-4382). The role of heparin/HSPG in the SFRP-1/Wnt binding and in Wnt signaling has been established (Haerry et al. (1997) Development 124:3055-3064; Binari et al. (1997) Development 124:2623-2632). Moreover, the positively charged hyaluronan-binding region of SFRP-1 may play an important role in binding to negatively charged matrix proteins like HSPG that results in increased local concentration of SFRP-1 and thus has a significant local antagonist effect on Wnt signaling. The matrix binding property of SFRP-1 may also result in the tissue-selective effect seen with SFRP. It should be noted that SFRP-1 is one of the key players of Wnt signaling in the bone as the deletion of this gene in mice lead to an increased bone mass in mice without any apparent non-skeletal phenotypic changes.

In the Examples below, the functional analysis of the SFRP-1 mutants and SFRP-1/SFRP-2 chimeras has clearly demonstrated that both the CRD and the netrin domains are necessary for optimal Wnt antagonist function. Recently, using a phage display library, a peptide that interacts with SFRP-1 has been identified. Utilizing the deletion mutants of SFRP-1 it has been shown that neither the CRD nor the netrin domain alone is sufficient for the binding of SFRP-1 to the peptide, however the last 71 amino acids of SFRP-1 are not necessary for its interaction with the peptide. Apparently, the contact points in both the CRD and netrin domains of SFRP-1 participated in the interaction with the peptide sequence (Chuman et al. (2004) Peptides 25:1831-1838). The Wnt antagonist function of SFRP-1 clearly requires the carboxyl-terminal sequence, and therefore it is tempting to speculate that the functional domains for Wnt antagonist function and peptide interaction may be different or overlapping. Thus the SFRP-1 mutants described in the present study will be a valuable tool to identify the critical motifs of SFRP-1 in its functions other than Wnt antagonism.

In defining the terms of the present invention, the term “bone formation” is the process of bone synthesis and mineralization. The term “bone-forming activity” is defined as performing the process of bone formation. The term “osteogenesis” is synonymous with the term bone formation, defined above. The term “bone growth” is the process of skeletal expansion. This process occurs by one of two ways: (1) intramembraneous bone formation arises directly from mesenchymal or bone marrow cells; (2) longitudinal or endochondral bone formation arises where bone forms from cartilage. The term “bone density” refers to the amount of bone tissue per a certain volume within bone. Low bone density is often associated with bone disorders, such as osteoporosis.

The term “secreted frizzled related proteins” or “SFRP” is a secreted receptor of the Wnt signaling pathway and exhibits a number of characteristics that make it a useful tool for studying cell growth and differentiation. “SFRP activity” refers to any of the biological activities of the native SFRP protein molecule, including, but not limited to, antagonism of the Wnt signaling pathway. The terms “secreted apoptosis related protein” and “SARP” are synonymous with the terms secreted frizzled related protein and SFRP, defined above. The terms hOB SFRP, FRP-1, FrzA and SARP-2 are synonymous with the term SFRP-1. The term “cysteine-rich domain” or “CRD” refers to a protein domain which has 10 conserved cysteines in its primary structure (amino acid sequence) that form five disulfide bridges. This domain is mainly (x-helical in structure.

SFRP-1 is one member of the SFRP family of proteins. SFRP-1 can have the amino acid sequence set forth in SEQ ID NO: 2, as encoded for by SEQ ID NO: 1. (The full gene is given in SEQ ID NO: 28.) SEQ ID NO: 2 is one amino acid different from the published sequence for human SFRP-1 (SEQ ID NO: 4): alanine instead of proline at position 174 (Melkonyan et al. (1997) Proc. Natl. Acad. Sci. USA 94:13636-13641). SEQ ID NO: 2 also differs by one amino acid from another reported SFRP-1 sequence (SEQ ID NO: 3) because SEQ ID NO: 2 has 314 amino acids, while GenBank™ Accession No. AAB61576 (SEQ ID NO: 3) has 313 amino acids (this difference is due to an additional alanine residue at position 13 in SEQ ID NO: 2).

The present invention encompasses methods using any SFRP proteins and mutated forms of the proteins found to be valuable for use in these methods. Also encompassed are the test compounds discovered through use of these methods.

The Wnt-signaling pathway may propagate a signal through several different mechanisms (Miller (2001) Genome Biol. 3:reviews3001.1-3001.15). Without being bound to any theory (an understanding of the mechanism is not necessary to practice the present invention, and the present invention is not limited to any particular mechanism), these include the Wnt/β-catenin, Wnt/Ca²⁺, and Wnt/polarity pathways. The mechanism currently understood to be most relevant to bone development is the Wnt/β-catenin pathway (also known as the canonical Wnt-signaling pathway). In this pathway, Wnt binds the Frizzled receptor which propagates a signal to inhibit the phosphorylation of β-catenin by glycogen synthase kinase-3 (GSK-3). Phosphorylated β-catenin would be ubiquitinated and degraded; however, by blocking GSK-3, β-catenin accumulates within the cell nucleus and activates T-cell factor/lymphoid enhancer factor (TCF/LEF) transcription factors, upregulating target genes. In the Wnt/Ca²⁺ pathway, the non-canonical Wnt pathway, Wnt activation of Fz results in a G-protein coupled response and the release of calcium into the cytoplasm from the endoplasmic reticulum. The increased calcium concentration activates protein kinase C (PKC) and calcium/calmodulin-regulated kinase II (CamKII). The Wnt/Ca²⁺ pathway antagonizes the Wnt/β-catenin pathway. Finally, the Wnt/polarity pathway regulates cytoskeletal organization and cellular axis determination.

“Wnt activity” refers to any of the biological activities of the native Wnt protein molecule and can be measured using a variety of methods, including measuring a change in one of the Wnt signaling pathways. One method involves the use of a TCF-luciferase assay. This assay uses a target reporter gene (luciferase) under the control of a TCF responsive element linked to a minimal promoter as described in the Examples section below. The TCF-luciferase assay can be used with any cell type. The effect of Wnt signaling on bone-related genes may be determined using the TCF-luciferase assay with bone cell types. Successful Wnt signaling produces a luciferase-mediated signal whereas a reduction in this signal indicates inhibition of the Wnt signaling pathway. Wnt activity may also be measured using transgenic animals, as described below. The effect of Wnt signaling on bone may be determined by measuring bone-related parameters. Further measurements may be performed using any of the pathway mechanisms described above or others that may be subsequently discovered. The measurement of Wnt activity as described is not limited to these examples.

“Incubating” a “sample” refers to, especially when used in terms of measuring Wnt activity, any method of having items, such that it may be determined if they interact with one another or are associated with a particular response. For example, “incubating a first sample comprising a wild-type SFRP and the test compound” means any method of bringing a wild-type SFRP and test compound together. The SFRP may be purified SFRP molecules used in a cell-free assay with purified test compound. Alternatively, the SFRP may be in a cell lysate assay with purified test compound. Furthermore, the SFRP may be encoded within DNA inserted within a cell that is exposed to the test compound. The SFRP and test compound may be determined to bind one another in a binding experiment or assay. The SFRP and test compound may be determined to be associated with a response such as Wnt activity. It is well appreciated within the art that many different systems may be used to test the interaction of two or more molecules, and the above examples by no means limit the invention.

The present invention identifies domains and specific amino acids of SFRPs that are important in the Wnt-signaling pathway. SFRPs inhibit the interaction of Wnt with Fz. It has been found that mutation or deletion of specific domains reduces the inhibition of SFRP for Wnt signaling, thus providing disinhibition, and allowing an increase in Wnt-signaling. An increase in Wnt-signaling promotes increased rate of bone formation, a decrease in osteoblast and osteoclast apoptosis, and an increase in osteoblast differentiation. The important domains include the CRD, netrin, hyaluronan domains, and the carboxy-terminal region. The specific amino acids found to be important are those amino acids corresponding to tyrosine 73 of SEQ ID NO: 2, the amino acids corresponding to lysine 228, 229, 230, 231, 234, 239, 240, 241, 244, and 245 of SEQ ID NO: 2, and additionally the amino acids corresponding to 73-86 of SEQ ID NO: 2, the amino acid corresponding to asparagine 173 of SEQ ID NO: 2, or the amino acids corresponding to 296- 314 of SEQ ID NO: 2, if the SFRP is SFRP-1.

The terms “proteins,” “peptides” and “polypeptides” are used interchangeably and are intended to include purified and recombinantly produced SFRP molecules containing amino acids linearly coupled through peptide bonds. The amino acids of this invention can be in the L or D form so long as the biological activity of the polypeptide is maintained. The SFRP proteins of this invention may also include proteins that are post-translationally modified by reactions that include, but are not limited to, glycosylation, acetylation, or phosphorylation. Such polypeptides also include analogs, alleles, and allelic variants that can contain amino acid derivatives or non-amino acid moieties that do not affect the biological or functional activity of the SFRP protein as compared to wild-type or naturally occurring protein. The term “amino acid” refers both to the naturally occurring amino acids and their derivatives, such as TyrMe and PheCl, as well as other moieties characterized by the presence of both an available carboxyl group and an amine group. Non-amino acid moieties that can be contained in such polypeptides include, for example, amino acid mimicking structures. Mimicking structures are those structures that exhibit substantially the same spatial arrangement of functional groups as amino acids but do not necessarily have both the amino and carboxyl groups characteristic of amino acids.

“Muteins” are protein or polypeptide “mutants” that have minor changes, i.e. “mutations,” in amino acid sequence caused, for example, by site-specific mutagenesis or other manipulations, by errors in transcription or translation, or which are prepared synthetically by rational design. These minor alterations result in amino acid sequences which may alter a biological activity or other characteristics of the protein or polypeptide compared to wild-type or naturally occurring polypeptide or protein.

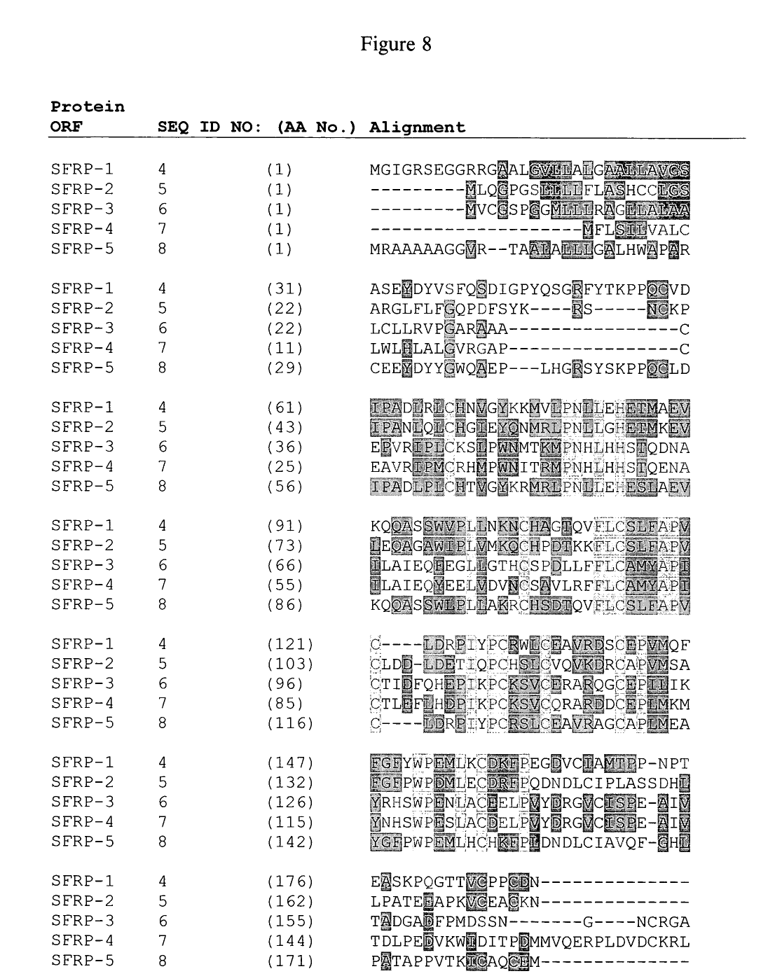

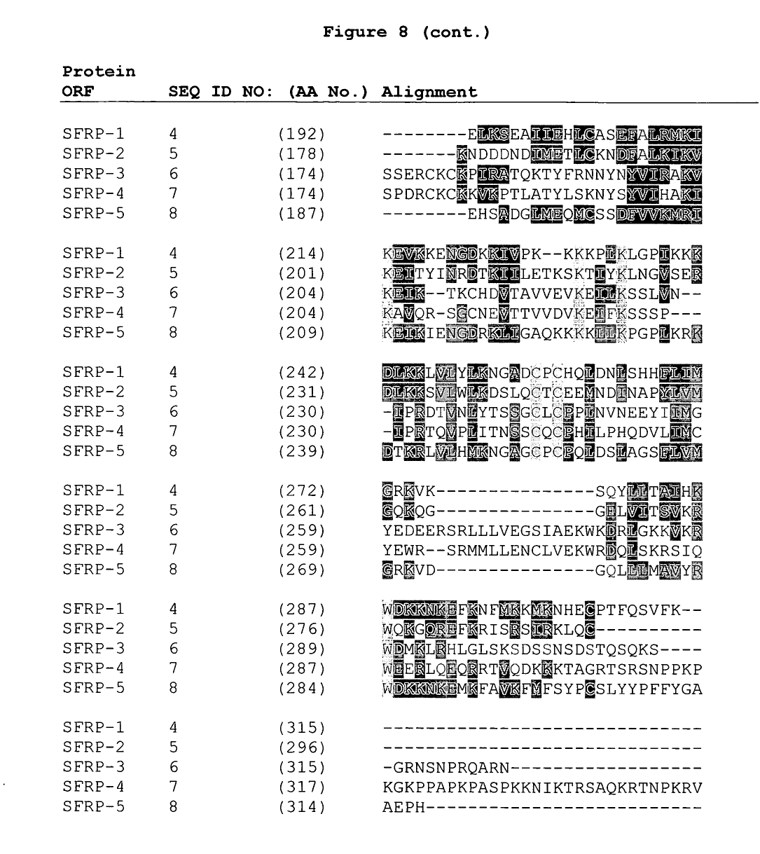

The phrases “corresponding to” and “corresponds to,” when applied to domains or amino acids within a protein, means the comparable domain or amino acid, respectively, within a protein using any SFRP as a reference. For example, SFRPs from separate animal species or individual SFRPs of one animal species may not have identical sequences. However, upon sequence alignment, for example as found in FIG. 8, it is appreciated by those who possess ordinary skill in the art that, even though aligned amino acids may have different numbering within their respective sequences, the domains or amino acids that align (even if not identical) are domains or amino acids that correspond to one another, respectively.

“Isolated,” when referring to an SFRP nucleic acid molecule, means separated from other cellular components normally associated with native or wild-type SFRP DNA or RNA intracellularly.

“Purified” when referring to an SFRP protein or polypeptide, is distinguishable from native or naturally occurring proteins or polypeptides because they exist in a purified state. These “purified” SFRP proteins or polypeptides, or any of the intended variations as described herein, shall mean that the compound or molecule is substantially free of contaminants normally associated with the compound in its native or natural environment. The terms “substantially pure” and “isolated” are not intended to exclude mixtures of polynucleotides or polypeptides with substances that are not associated with the polynucleotides or polypeptides in nature.

A “purification tag” is any molecular moiety added to a peptide or protein to aid in the purification process. Purification tags well known in the art include, but are not limited to, antibody recognition tags (affinity tags, e.g. myc epitope tag), histidine (His) tags, streptavidin binding peptide (SBP) tags, maltose binding protein (MBP) tags, and glutathione S-transferase (GST) tags.

“Native” SFRP polypeptides, proteins, or nucleic acid molecules refer to those SFRPs recovered from a source occurring in nature or “wild-type.”

The term “small molecule” refers to a compound that has a molecular weight of less than about 2000 Daltons, less than about 1000 Daltons, or less than about 500 Daltons. Small molecules, without limitation, may be, for example, nucleic acids, peptides, polypeptides, peptide nucleic acids, peptidomimetics, carbohydrates, lipids, or other organic (carbon containing) or inorganic molecules and may be synthetic or naturally occurring or optionally derivatized. Such small molecules may be a therapeutically deliverable substance or may be further derivatized to facilitate delivery or targeting.

The term “nucleic acid” as it relates to the SFRP described herein means single and double-stranded DNA, cDNA, genome-derived DNA, and RNA, as well as the positive and negative strand of the nucleic acid that are complements of each other, including anti-sense RNA. A “nucleic acid molecule” is a term used interchangeably with “polynucleotide” and each refers to a polymeric form of nucleotides of any length, either ribonucleotides or deoxyribonucleotides, or analogs thereof. It also includes known types of modifications, for example, labels which are known in the art (e.g., Sambrook et al., (1989) infra.), methylation, “caps,” substitution of one or more of the naturally occurring nucleotides with an analog, internucleotide modifications such as, for example, those with uncharged linkages (e.g., methyl carbamate, etc.), those containing pendant moieties, such as for example, proteins (including, e.g., nuclease, toxins, antibodies, signal peptides, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide. The polynucleotide can be chemically or biochemically modified or contain non-natural or derivatized nucleotide bases. The nucleotides may be complementary to the mRNA encoding the polypeptides. These complementary nucleotides include, but are not limited to, nucleotides capable of forming triple helices and antisense nucleotides. Recombinant polynucleotides comprising sequences otherwise not naturally occurring are also provided by this invention, as are alterations of wild-type polypeptide sequences, including but not limited to, those due to deletion, insertion, substitution of one or more nucleotides or by fusion to other polynucleotide sequences.

An SFRP polynucleotide is said to “encode” an SFRP polypeptide if, in its native state or when manipulated by methods well-known to those skilled in the art, it can be transcribed and/or translated to produce a polypeptide or mature protein. Thus, the term polynucleotide shall include, in addition to coding sequences, processing sequences and other sequences that do not code for amino acids of the mature protein. The anti-sense strand of such a polynucleotide is also said to encode the sequence.

The term “recombinant” polynucleotide or DNA refers to a polynucleotide that is made by the combination of two otherwise separated segments of sequence accomplished by the artificial manipulation of isolated segments of DNA by genetic engineering techniques or by chemical synthesis. In so doing, one may join together DNA segments of desired functions to generate a desired combination of functions.

An “analog” of an SFRP DNA, RNA or polynucleotide refers to a macromolecule resembling naturally occurring polynucleotides in form and/or function (particularly in the ability to engage in sequence-specific hydrogen bonding to base pairs on a complementary polynucleotide sequence) but which differs from DNA or RNA in, for example, the possession of an unusual or non-natural base or an altered backbone. See, for example, Uhlmann et al. (1990) Chem. Rev. 90:543-584.

“Hybridization” refers to hybridization reactions that can be performed under conditions of different “stringency”. Conditions that increase the stringency of a hybridization reaction are widely known and published in the art: see, for example, Sambrook et al., infra. Examples of relevant conditions include (in order of increasing stringency): incubation temperatures of 25° C., 37° C., 50° C., and 68° C.; buffer concentrations of 10×SSC, 6×SSC, 1×SSC, 0.1×SSC (where SSC is 0.15 M NaCl and 15 mM citrate buffer) and their equivalent using other buffer systems; formamide concentrations of 0%, 25%, 50%, and 75%, incubation times from 5 minutes to 24 hours and washes of increasing duration, increasing frequency, or decreasing buffer concentrations.

“Tₘ” is the temperature in degrees Centigrade at which 50% of a polynucleotide duplex made of complementary strands hydrogen bonded in an antiparallel direction by Watson-Crick base paring dissociates into single strands under the conditions of the experiment. Tₘ may be predicted according to standard formulas, for example:
Tₘ=81.5+16.6log[Na⁺]+0.41(% G/C)−0.61(% F)−600/L
where [Na⁺] is the cation concentration (usually sodium ion) in mol/L; (% G/C) is the number of G and C residues as a percentage of total residues in the duplex; (% F) is the percent formamide in solution (wt/vol); and L is the number of nucleotides in each strand of the duplex.

A “stable duplex” of polynucleotides, or a “stable complex” formed between any two or more components in a biochemical reaction, refers to a duplex or complex that is sufficiently long lasting to persist between the formation of the duplex or complex, and its subsequent detection. The duplex or complex must be able to withstand whatever conditions exist or are introduced between the moment of formation and the moment of detection, these conditions being a function of the assay or reaction which is being performed. Intervening conditions which may optionally be present and which may dissociate a duplex or complex include washing, heating, adding additional solutes or solvents to the reaction mixture (such as denaturants), and competing with additional reacting species. Stable duplexes or complexes may be irreversible or reversible, but must meet the other requirements of this definition. Thus, a transient complex may form in a reaction mixture, but it does not constitute a stable complex if it dissociates spontaneously or as a result of a newly imposed condition or manipulation introduced before detection.

When stable duplexes form in an antiparallel configuration between two single-stranded polynucleotides, particularly under conditions of high stringency, the strands are essentially “complementary.” A double-stranded polynucleotide can be “complementary” to another polynucleotide, if a stable duplex can form between one of the strands of the first polynucleotide and the second. A complementary sequence predicted from the sequence of single stranded polynucleotide is the optimum sequence of standard nucleotides expected to form hydrogen bonding with the single-stranded polynucleotide according to generally accepted base-pairing rules.

A “sense” strand and an “antisense” strand when used in the same context refer to single-stranded SFRP polynucleotides which are complementary to each other. They may be opposing strands of a double-stranded polynucleotide, or one strand may be predicted from the other according to generally accepted base-pairing rules. Unless otherwise specified or implied, the assignment of one or the other strand as “sense” or “antisense” is arbitrary.

A linear sequence of SFRP nucleotides is “identical” to another linear sequence if the order of nucleotides in each sequence is the same, and occurs without substitution, deletion, or material substitution. It is understood that purine and pyrimidine nitrogenous bases with similar structures can be functionally equivalent in terms of Watson-Crick base-pairing; and the inter-substitution of like nitrogenous bases, particularly uracil and thymine, or the modification of nitrogenous bases, such as by methylation, does not constitute a material substitution so long as the substitution does not alter hydrogen bonding between the bases. An RNA and a DNA polynucleotide have identical sequences when the sequence for the RNA reflects the order of nitrogenous bases in the polyribonucleotide, the sequence for the DNA reflects the order of nitrogenous bases in the polydeoxyribonucleotide, and the two sequences satisfy the other requirements of this definition. Where at least one of the sequences is a degenerate oligonucleotide comprising an ambiguous residue, the two sequences are identical if at least one of the alternative forms of the degenerate oligonucleotide is identical to the sequence with which it is being compared. For example, AYAAA is identical to ATAAA, if AYAAA is a mixture of ATAAA and ACAAA and AYAAA is being compared to ATAAA.

When comparison is made between polynucleotides, it is implicitly understood that complementary strands are easily generated, and the sense or antisense strand is selected or predicted that maximizes the degree of identity between the polynucleotides being compared. For example, where one or both of the polynucleotides being compared is double-stranded, the sequences are identical if one strand of the first polynucleotide is identical with one strand of the second polynucleotide. Similarly, when a polynucleotide probe is described as identical to its target, it is understood that it is the complementary strand of the target that participates in the hybridization reaction between the probe and the target.

A linear sequence of nucleotides is “essentially identical” or the “equivalent” to another linear sequence if both sequences are capable of hybridizing to form duplexes with the same complementary polynucleotide. It should be understood, although not always explicitly stated, that when Applicants refer to a specific nucleic acid molecule, its equivalents are also intended. Sequences that hybridize under conditions of greater stringency are one embodiment. It is understood that hybridization reactions can accommodate insertions, deletions, and substitutions in the nucleotide sequence. Thus, linear sequences of nucleotides can be essentially identical even if some of the nucleotide residues do not precisely align. Sequences that align more closely to the invention disclosed herein are another embodiment. Generally, a polynucleotide region of about 25 residues is essentially identical to another region if the sequences are at least about 85% identical, at least about 90% identical, at least about 95% identical, or 100% identical. A polynucleotide region of 40 residues or more will be essentially identical to another region, after alignment of homologous portions, if the sequences are at least about 85% identical, at least about 90% identical at least 95% identical, or 100% identical.

The phrases “corresponding to” and “corresponds to,” when applied to nucleic acids, means the comparable base within a nucleic acid molecule using any SFRP as a reference. For example, SFRPs from separate animal species or individual SFRPs of one animal species may not have identical sequences. However, upon sequence alignment, it is appreciated by those who possess ordinary skill in the art that, even though aligned bases may have different numbering within their respective sequences, the bases that align are bases that correspond to one another.

In determining whether polynucleotide sequences are essentially identical, a sequence that preserves the functionality of the polynucleotide with which it is being compared is one embodiment. Functionality can be determined by different parameters. For example, if the polynucleotide is to be used in reactions that involve hybridizing with another polynucleotide, then preferred sequences are those which hybridize to the same target under similar conditions. In general, the Tₘ of a DNA duplex decreases by about 10C for every 1% decrease in sequence identity for duplexes of 200 or more residues; or by about 50° C. for duplexes of less than 40 residues, depending on the position of the mismatched residues (see, e.g. Meinkoth and Wahl (1984) Anal. Biochem. 138:267-284). Essentially identical or equivalent sequences of about 100 residues will generally form a stable duplex with each other's respective complementary sequence at about 20° C. less than Tₘ, at about 15° C. less, at about 10° C. less, at about 5° C. less, at about Tₘ. In another example, if the polypeptide encoded by the polynucleotide is an important part of its functionality, then preferred sequences are those which encode identical or essentially identical polypeptides. Thus, nucleotide differences which cause a conservative amino acid substitution are one embodiment; nucleotide differences which cause non-conservative amino acid substitutions are another embodiment; nucleotide differences which do not alter the amino acid sequence are an embodiment while identical nucleotides are yet another embodiment. Insertions or deletions in the polynucleotide that result in insertions or deletions in the polypeptide are embodiments whereas those that result in the down-stream coding regions being rendered out of phase are another embodiment; polynucleotide sequences comprising no insertions or deletions are another embodiment. The relative importance of hybridization properties and the encoded polypeptide sequence of a polynucleotide depend on the application of the invention.

A polynucleotide has the same characteristics or is the equivalent of another polynucleotide if both are capable of forming a stable duplex with a particular third polynucleotide under similar conditions of maximal stringency. Preferably, in addition to similar hybridization properties, the polynucleotides also encode essentially identical polypeptides.

“Conserved” residues of a polynucleotide sequence are those residues that occur unaltered in the same position of two or more related sequences being compared. Residues that are relatively conserved are those that are conserved amongst more related sequences than residues appearing elsewhere in the sequences.

As used herein, a “degenerate” oligonucleotide sequence is a designed sequence derived from at least two related originating polynucleotide sequences as follows: the residues that are conserved in the originating sequences are preserved in the degenerate sequence, while residues that are not conserved in the originating sequences may be provided as several alternatives in the degenerate sequence. For example, the degenerate sequence AYASA may be assigned from originating sequences ATACA and ACAGA, where Y is C or T and S is C or G. Y and S are examples of “ambiguous” residues. A degenerate segment is a segment of a polynucleotide containing a degenerate sequence.

It is understood that a synthetic oligonucleotide comprising a degenerate sequence can be a mixture of closely related oligonucleotides sharing an identical sequence, except at the ambiguous positions. Such a mixture of all possible combinations of nucleotides may be produced by synthetic methods. Each of the oligonucleotides in the mixture is referred to as an “alternative form.”

A polynucleotide “fragment” or “insert” as used herein generally represents a sub-region of the full-length form, but the entire full-length polynucleotide may also be included.

Different polynucleotides “relate” to each other if one is ultimately derived from another. For example, messenger RNA relates to the gene from which it is transcribed. cDNA relates to the RNA from which it has been produced, such as by a reverse transcription reaction, or by chemical synthesis of a DNA based upon knowledge of the RNA sequence or the coding sequence of genomic DNA. cDNA also relates to the coding sequence of the gene that encodes the RNA. Polynucleotides also “relate” to each other if they serve a similar function, such as encoding a related polypeptide in different species, strains or variants that are being compared.

The term “upstream” refers to nucleic acid base(s) or base pair(s) that are 5′ to the reference nucleic acid base(s) within a nucleic acid molecule, the 5′ determined using the sense strand, or the strand derived from the sense strand, if the nucleic acid molecule is double stranded.

A “probe” when used in the context of SFRP polynucleotide manipulation refers to an oligonucleotide that is provided as a reagent to detect a target potentially present in a sample of interest by hybridizing with the target. Usually, a probe will comprise a label or a means by which a label can be attached, either before or subsequent to the hybridization reaction. Suitable labels include, but are not limited to, radioisotopes, fluorochromes, chemiluminescent compounds, dyes, and proteins, including enzymes.

A “primer” is an oligonucleotide, generally with a free 3′-OH group, that binds to a target potentially present in a sample of interest by hybridizing with the target, and thereafter promotes polymerization of a polynucleotide complementary to the target.

Processes of producing replicate copies of the same polynucleotide, such as PCR or gene cloning, are collectively referred to herein as “amplification” or “replication”. For example, single or double-stranded DNA may be replicated to form another DNA with the same sequence. RNA may be replicated, for example, by an RNA-directed RNA polymerase, or by reverse-transcribing the DNA and then performing a PCR. In the latter case, the amplified copy of the RNA is a DNA with the identical sequence.

Elements within a gene include, but are not limited to, promoter regions, enhancer regions, repressor binding regions, transcription initiation sites, ribosome binding sites, translation initiation sites, protein encoding regions, introns and exons, and termination sites for transcription and translation. An “antisense” copy of a particular polynucleotide refers to a complementary sequence that is capable of hydrogen bonding to the polynucleotide and can therefore, be capable of modulating expression of the polynucleotide. These are DNA, RNA or analogs thereof, including analogs having altered backbones, as described above. The polynucleotide to which the antisense copy binds may be in single-stranded form or in double-stranded form.

As used herein, the term “operatively linked” means that the DNA molecule is positioned relative to the necessary regulation sequences, e.g., a promoter or enhancer, such that the promoter will direct transcription of RNA off the DNA molecule in a stable or transient manner.

“Vector” means a self-replicating nucleic acid molecule that transfers an inserted nucleic acid molecule into and/or between host cells. The term is intended to include vectors that function primarily for the replication of nucleic acid and expression vectors that function for transcription and/or translation of the DNA or RNA. Also intended are vectors that provide more than one of the above functions.

“Host cell” is intended to include any individual cell or cell culture that can be or have been recipients for vectors or the incorporation of exogenous nucleic acid molecules and/or proteins. It also is intended to include progeny of a single cell, and the progeny may not necessarily be completely identical (in morphology or in genomic or total DNA complement) to the original parent cell due to natural, accidental, or deliberate mutation.

An “antibody” is an immunoglobulin molecule capable of binding an antigen. As used herein, the term encompasses not only intact immunoglobulin molecules, but also anti-idiotypic antibodies, mutants, fragments, fusion proteins, humanized proteins and modifications of the immunoglobulin molecule that comprise an antigen recognition site of the required specificity.

An “antibody complex” is the combination of antibody (as defined above) and its binding partner or ligand.

A “suitable cell” for the purposes of this invention is one that includes, but is not limited to, a cell expressing the SFRP, e.g., a bone marrow cell, preferentially an hOB cell.

A “biological equivalent” of a nucleic acid molecule is defined herein as one possessing essential identity with the reference nucleic acid molecule. A fragment of the reference nucleic acid molecule is one example of a biological equivalent.

A “biological equivalent of an SFRP polypeptide or protein” is one that retains the same characteristic as the reference protein or polypeptide. This definition includes fragments of the reference protein or polypeptide that retain the same characteristic as the reference protein or polypeptide.

The SFRP proteins and polypeptides also can be obtained by chemical synthesis using a commercially available automated peptide synthesizer such as those manufactured by Applied Biosystems, Inc., (Foster City, Calif.) Model 430A or 431A, and the amino acid sequence provided in SEQ ID NO: 2. The synthesized protein or polypeptide can be precipitated and further purified, for example by high performance liquid chromatography (HPLC). Accordingly, this invention also provides a process for chemically synthesizing the proteins of this invention by providing the sequence of the protein (e.g., SEQ ID NO: 2) and reagents, such as amino acids and enzymes and linking together the amino acids in the proper orientation and linear sequence.

Alternatively, the proteins and polypeptides can be obtained by well-known recombinant methods as described, for example, in Sambrook et al. ((1989) Molecular Cloning: A Laboratory Manual. 2d ed. Cold Spring Harbor Laboratory) using, for example, the host cell and vector systems described and exemplified in U.S. Patent Application, Pub. No. 2004/0115195 (U.S. Ser. No. 10/666,851). An SFRP, analog, mutein or fragment thereof, may be produced by growing a host cell containing a nucleic acid molecule encoding the desired protein, the nucleic acid being operatively linked to a promoter of RNA transcription. The desired protein may be introduced into the host cell by use of a gene construct which contains a promoter and termination sequence for the nucleic acid sequence of the desired protein. The host cell is grown under suitable conditions such that the nucleic acid is transcribed and translated into protein. In a separate embodiment, the protein is further purified.

In accordance with the present invention, there may be employed conventional molecular biology, microbiology, recombinant DNA, immunology, cell biology and other related techniques within the skill of the art. See, e.g., Sambrook et al. (2001) Molecular Cloning: A Laboratory Manual. 3^{rd} ed. Cold Spring Harbor Laboratory Press: Cold Spring Harbor, N.Y.; Sambrook et al. (1989) Molecular Cloning: A Laboratory Manual. 2^{nd} ed. Cold Spring Harbor Laboratory Press: Cold Spring Harbor, N.Y.; Ausubel et al. eds. (2006) Current Protocols in Molecular Biology. John Wiley and Sons, Inc.: Hoboken, N.J.; Bonifacino et al. eds. (2006) Current Protocols in Cell Biology. John Wiley and Sons, Inc.: Hoboken, N.J.; Coligan et al. eds. (2006) Current Protocols in Immunology, John Wiley and Sons, Inc.: Hoboken, N.J.; Coico et al. eds. (2006) Current Protocols in Microbiology, John Wiley and Sons, Inc.: Hoboken, N.J.; Coligan et al. eds. (2006) Current Protocols in Protein Science, John Wiley and Sons, Inc.: Hoboken, N.J.; Enna et al. eds. (2006) Current Protocols in Pharmacology John Wiley and Sons, Inc.: Hoboken, N.J.; Hames et al. eds. (1999) Protein Expression: A Practical Approach. Oxford University Press: Oxford; Freshney (2000) Culture of Animal Cells: A Manual of Basic Technique. 4^{th} ed. Wiley-Liss; among others. The Current Protocols listed above are updated several times every year.

The proteins of this invention also can be combined with various liquid phase carriers, such as sterile or aqueous solutions, pharmaceutically acceptable carriers, suspensions and emulsions. Examples of non-aqueous solvents include propyl ethylene glycol, polyethylene glycol and vegetable oils. When used to prepare antibodies, the carriers also can include an adjuvant that is useful to nonspecifically augment a specific immune response. A skilled artisan can easily determine whether an adjuvant is required and select one. However, for the purpose of illustration only, suitable adjuvants include, but are not limited to, Freund's Complete and Incomplete, mineral salts and polynucleotides.

### Therapeutic Applications for SFRPs/SARPs

The methods of the present invention allow for identification of compounds that target the Wnt-antagonizing function of SFRP by way of a particular amino acid or amino acids. In one embodiment, the compounds identified can increase the activity of Wnt by inhibiting the interaction of SFRP with Wnt, thus inhibiting the inhibitor (disinhibition).

Wnts may also be utilized in tissue regeneration. For example, since FrzB-1 stimulated ectopic chondrogenic activity in vivo, it could be used to accelerate fracture repair or the healing of joints after hip and knee replacement (see, International Patent Publication No. WO 98/16641, incorporated herein by reference in its entirety). Finally, because SFRPs/SARPs appear to control apoptosis, these proteins could also be utilized to treat a variety of degenerative diseases including neurodegeneration, myodegeneration and osteodegeneration disorders.

Pharmaceutical compositions of the test compounds discovered using the present invention are useful for treating or preventing osteodegeneration disorders such as osteoporosis and the bone resorptive disease, Paget's disease. For instance, when SFRP-1 expression and/or activity are abolished in vivo (e.g., in transgenic mice) bone density is increased, resulting in a delay of age-dependent bone loss (see WO 01/19855, incorporated herein by reference in its entirety). These effects correlate generally with an increased rate of bone formation, a decrease in osteoblast and osteoclast apoptosis, and an increase in osteoblast differentiation. Disruption of the fine balance between the differentiation of new osteoclast and osteoblast cells and the timing of cell death by apoptosis are thought to be important mechanisms behind bone loss disorders. Thus, therapeutic agents that alter the prevalence of apoptosis in osteoblasts and/or osteoclasts are useful and desirable to correct the imbalance in cell numbers that is the basis of diminished bone mass and increased risk of fractures in osteoporosis. For review, see, Manolagas (2000) Endocr. Rev. 21:115-137; and Weinstein and Manolagas (2000) Am. J. Med. 108:153-164.

For example, in one embodiment, test compounds discovered using the present invention may be used to prevent an osteodegenerative disorder, e.g., in an individual who may not have a bone degeneration disorder but who has or is suspecting of being susceptible to such a disorder. In another embodiment, the test compounds discovered using the present invention can be used to prevent Type II or “senile” osteoporosis. As a particular example, and not by way of limitation, test compounds discovered using the present invention may be administered to a juvenile, adolescent or young adult.

Altering the activity of SFRP-1 does not produce any significant side effects, e.g., on cortical bone and non-skeletal tissues, in body or organ weight; serum calcium, phosphorus, bone-alkaline phosphatase or osteocalcin levels; urinary deoxy-pyridinoline cross-link levels; total body bone mineral density (BMD), bone mineral content and percentage body fat; or cortical BMD (see U.S. Patent Application Pub. No. 2004/0115195, U.S. Ser. No. 10/666,851). Even though the inhibition of SFRP-1 may increase bone density, it does not alter skeletal development. Consequently, therapeutic methods and compositions that specifically inhibit SFRP-1 activity and/or expression are expected to have very few or even no detrimental side effects.

Alternatively the test compounds discovered through the use of the methods of the present invention can be used to treat diseases. Such diseases may be treated by disrupting or decreasing SFRP activity by means of, for example, antibodies, antisense SFRP nucleotides, siRNAs or shRNAs that inhibit SFRP expression, or small molecule inhibitors that disrupt or decrease SFRP activity and/or expression. In particular, the present invention identifies specific amino acids of SFRPs that are important in decreasing SFRP activity.

### Pharmaceutical Compositions

A “pharmaceutical composition” is intended to include antibodies, small molecules, or test compounds that are targeted to particular amino acids of SFRP for decreasing or blocking SFRP activity as the active agent with a carrier, inert or active, making the composition suitable for diagnostic or therapeutic use in vitro, in vivo, or ex vivo.

This invention also provides compositions containing a test compound discovered using the present invention and an acceptable solid or liquid, carrier buffer, or diluent. An effective amount of one or more active ingredient is used which is sufficient to accomplish the desired regulatory effect on a bone-forming activity or apoptosis activity. An effective amount can be determined by conventional dose-response curves for the desired activity. When the compositions are used pharmaceutically, they are combined with a “pharmaceutically acceptable carrier” for diagnostic and therapeutic use. The formulation of such compositions is well known to persons skilled in this field. Pharmaceutical compositions of the invention may comprise one or more additional active components and include a pharmaceutically acceptable carrier. The additional active component may be provided to work in combination with an active component based on one or more SFRPs, as described above. In alternative embodiments, the additional active component is added because it works on the same disease or disorder as SFRPs but by a different mode of action from those actives based on SFRPs, or the additional active may work on other diseases or disorders present in a human or animal.

Suitable pharmaceutically acceptable carriers and/or diluents include any and all conventional solvents, dispersion media, fillers, solid carriers, aqueous solutions, coatings, antibacterial and antifungal agents, isotonic and absorption delaying agents, and the like with which the compound is administered. The term “pharmaceutically acceptable carrier” refers to a carrier that does not cause an allergic reaction or other untoward effect in patients to whom it is administered. As used herein, the term “pharmaceutically acceptable” means approved by a regulatory agency of the Federal or state government or listed in the U.S. Pharmacopeia or other generally recognized pharmacopeia for use in animals, and more particularly, in humans. Specific, suitable pharmaceutically acceptable carriers include, but are not limited to, for example, one or more of water, saline, phosphate buffered saline, dextrose, glycerol, ethanol and the like, as well as combinations thereof. Water or aqueous solution saline solutions and aqueous dextrose and glycerol solutions may be employed as carriers, particularly for injectable solutions. Pharmaceutically acceptable carriers may further comprise minor amounts of auxiliary substances such as wetting or emulsifying agents, preservatives or buffers, which enhance the shelf life or effectiveness of one or more of the active components of the composition. The use of such media and agents for pharmaceutically active substances is well known in the art and suitable pharmaceutical carriers are described in “Remington's Pharmaceutical Sciences” by E. W. Martin. Except insofar as any conventional media or agent is incompatible with the active ingredient, use thereof in immunogenic compositions of the present invention is contemplated.

These pharmaceutical compositions also can be used for the preparation of medicaments for the diagnosis and treatment of pathologies associated with neurodegenerative (i.e., Huntington's disease, Alzheimer's disease, and spinal cord injuries), myodegenerative (i.e., muscular dystrophy, myasthenia gravis, myotonic myopathies) and osteodegenerative disorders (i.e., osteoporosis). These compositions can also be used for the preparation for medicaments for the diagnosis and treatment of diseases such as Paget's disease, osteosclerosis, osteogenesis imperfecta, fibrous dysplasia, hypophosphatasia and osteopetrosis.

In certain embodiments, antibodies that bind all or a portion of an SFRP protein are employed in the composition to treat of any of the above diseases or disorders, particularly those targeted to one or more domain selected from the group consisting of the cysteine rich domain (CRD), the netrin domain, and the hyaluronan domain or one or more of the amino acids corresponding to tyrosine 73 of SEQ ID NO: 2, the amino acids corresponding to lysine 228, 229, 230, 231, 234, 239, 240, 241, 244, and 245 of SEQ ID NO: 2, and additionally the amino acids corresponding to 73-86 of SEQ ID NO: 2, or the amino acid corresponding to asparagine 173 of SEQ ID NO: 2, or the amino acids corresponding to 296-314 of SEQ ID NO: 2, if the SFRP is SFRP-1.

Antibodies for in vivo use may recognize a topological or conformational epitope present on the native SFRP molecule. The antibodies contemplated by the present invention may or may not recognize denatured SFRP or SFRP fragments. Polyclonal and monoclonal antibodies can be prepared by conventional methods, e.g., by immunization with SFRP protein or a mutant thereof. Alternatively, an antibody is raised against an amino acid sequence (a) that is specific to an SFRP polypeptide/protein (or proteins) and (b) that is also more likely to be antigenic. One can select a sequence specific for an SFRP protein by performing sequence analysis and using any conventional programs for sequence alignment and sequence comparisons. An amino acid sequence that is hydrophilic at one or more ends, or at both ends, is generally favored for raising antibodies. In addition to employing amino acids that are hydrophilic, in some embodiments the hydrophilic amino acids are also basic (non-acidic). One can also employ any amino acid that increases antigenicity. For example, often prolines are employed in the center portion of the sequence. Antigenicity can be measured by an increase in the decrease in the amount of antibody that is produced when generating antibodies against an initial test sequence, which is specific to SFRP protein(s).

In certain embodiments of the present invention, the antibody is raised against a sequence comprising at least 8 consecutive amino acids of an SFRP protein(s), or a sequence comprising at least 10 consecutive amino acids of an SFRP protein(s). In other embodiments, the antibody is raised against amino acid sequence comprising about 15 to about 30 amino acids. In other embodiments, the antibody is raised against a sequence comprising amino acids 68-86, or preferably 73-86, of an SFRP-1 protein, a polypeptide having the amino acid sequence set forth in SEQ ID NO: 2, or sequence variations thereof.

The compositions of the test compounds discovered using the methods of the present invention can be administered to an individual in need of facilitated neural, muscle, cartilage, and bone growth by numerous routes, including, but not limited to, intravenous, subcutaneous, intramuscular, intrathecal, intracranial and topical. The composition may be administered directly to an organ or to organ cells by in vivo or ex vivo methods.

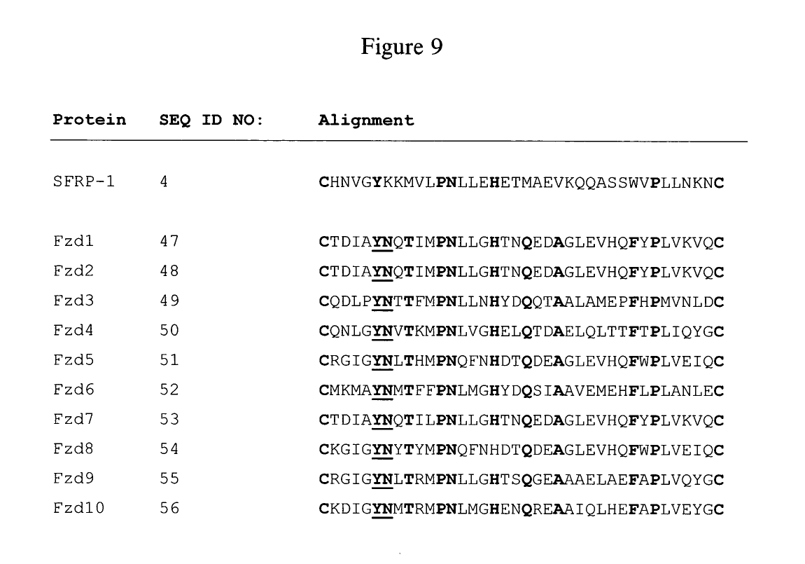

The test compounds can be tailored to bind SFRPs and not Frizzleds. The netrin and hyaluronan domains of the SFRPs do not share homology with the Frizzleds. Therefore, test compounds that target these domains, or amino acids within them, of SFRP are unlikely to interact with Frizzleds. The CRDs do share homology, as is shown in FIG. 9 for SFRP-1 and several Frizzleds around the conserved tyrosine 73. There is variability within the CRDs of the SFRPs and FZDs where test molecules may be designed or experimentally determined such that the test molecules have a higher affinity and/or greater selectivity for SFRPs than for Frizzleds.

Pharmaceutical compositions may be in soluble or microparticular form or may be incorporated into microspheres or microvesicles, including micelles and liposomes.

### Screening Methods

Screening methods may be devised wherein the amount of a test compound bound to an SFRP may be determined. In such a screening method, there may be two samples. In one, a molecular species, a “test compound,” and wild-type SFRP may be incubated together, and, separately in another, the test compound and an SFRP mutant may be incubated together. The test compound may be, but is not limited to, a small molecule, polypeptide, or nucleic acid or may be a gene that is knocked-out or knocked-down in a cell. The amount of compound bound to each may then be determined using any of various methods known to those of ordinary skill in the art. These could include, but are not limited to, using fluorescence (such as in a tryptophan fluorescence quenching assay or FRET-based assay), nuclear magnetic resonance, and chromatography. Tryptophan fluorescence quenching assays are generally used to measure the ability of compounds to bind and alter protein conformation. Compounds that bind to a protein may give the result of reduced fluorescence. This assay has been shown to be successful in binding studies.

Screening methods may be devised based on the specific domains or amino acids of SFRP found to be important in inhibition of the Wnt signaling pathway. As non-limiting examples, the described assay methods may be used to determine a change in Wnt signaling in the presence or absence of a test compound. Any other assay method may be used provided that it shows a change in Wnt signaling due to the presence or absence of a test compound. Such assays may be, but are not limited to, activity or binding assays. Non-limiting examples include methods as described in the Summary section. Furthermore, in addition to using two samples (wild-type SFRP in the presence of a test compound and a mutant SFRP in the presence of the test compound), assays may be devised such that the amount of binding of SFRP to Wnt, or the activity of Wnt signaling, may be compared to samples of the wild-type SFRP and/or the mutant SFRP in the absence of the test compound. This comparison may provide additional information found useful in determining compounds that target the SFRP.

Suitable cells can be used for preparing diagnostic assays, for the expression of SFRPs or for preparing nucleotide-based diagnostic kits. The cells may be made or derived from yeast, bacteria, fungi, or viruses. In certain embodiments, the cells are hOB cells, in particular a novel immortalized pre-osteocytic cell line referred to as hOB-01-C1-PS-09 cells (which are deposited with American Type Culture Collection in Manassas, Va. with the designation PTA-785), and osteoblast cells having the identifying characteristics of hOB-01-C1-PS-09 cells as well as osteoblast cells made therefrom, e.g. progeny.

Agents according to the present invention may be identified by screening in high-throughput assays, including, without limitation, cell-based or cell-free assays. It will be appreciated by those skilled in the art that different types of assays can be used to detect different types of agents. Several methods of automated assays have been developed in recent years so as to permit screening of tens of thousands of compounds in a short period of time (see, e.g., U.S. Pat. Nos. 5,585,277; 5,679,582; and 6,020,141).

### Transgenic Animals Demonstrate Relationship between SFRP and Bone Formation

A compound that inhibits osteoblast/osteocyte apoptosis would conceivably be an anabolic bone agent by prolonging the lives of these cells and thereby either increasing the amount of bone matrix that is synthesized and mineralized and/or maintaining the integrity of the bone. In order to test this hypothesis and determine if SFRP-1/FRP-1/SARP-2 affects the skeleton, SFRP-1 −/− mice were prepared in WO 01/19855 (see also Wattler et al. (1999) BioTechniques 26:1150-1160).

Deleting the SFRP-1/FRP-1/SARP-2 gene from mice would be akin to inhibiting its function with a compound, and this process allows validation of this gene/protein as a potential pharmaceutical target for osteoporosis.

The SFRP-1 knock-out mice were generated in WO 01/19855 by substituting exon 1 of the mouse SFRP-1 gene with β-galactosidase reporter gene/neomycin resistance gene expression cassette. Northern blot analysis of poly A+RNA isolated from either female of male kidneys (age 16-18 weeks) demonstrated high levels of SFRP-1 mRNA expression (4.4 kb) in the wild-type (WT) control mice, but a complete absence of gene expression in the knock-out (KO) mice.

Micro computerized tomography (micro-CT) was used in WO 01/19855 to characterize the trabecular bone architecture of the distal femurs from male and female wild-type control (+/+) and knock-out (−/−) mice (for a review of this technique, see Genant et al. (1999) Bone 25: 149-152 and Odgaard (1997) Bone 20:315-328). In the 20 week old males, the −/− mice had 31% more trabecular bone volume (BV/TV) and an 8% increase in trabecular thickness (Tb. Th.) when compared to the +/+ control mice. In the 26-27 week old females, the −/− mice had a 91% increase in trabecular connectivity density (Conn. Den.), a 16% increase in trabecular number (Tb. N.) and a 16% decrease in trabecular spacing (Tb. Sp.) when compared to the +/+ control mice.

Thus, these results demonstrate that deletion of the SFRP-1 gene in mice leads to increased parameters of trabecular bone formation (P. J. Meunier (1995) Bone Histomorphometry, in Osteoporosis: Etiology, Diagnosis, and Management. 2 ed. B. L. Riggs and L. J. Meltonlil, eds. Lippincott-Raven: Philadelphia, pages 299-318).

Transgenic and/or non-genetically modified animals may be used for methods of screening compounds which may be of pharmaceutical interest. Non-limiting examples of animals would be SFRP +/+, SFRP −/−, or a transgenic animal genetically modified such that one or more of an amino acid selected from the group consisting of the amino acid corresponding to tyrosine 73 of SEQ ID NO: 2, the amino acids corresponding to lysine 228, 229, 230, 231, 234, 239, 240, 241, 244, and 245 of SEQ ID NO: 2, and additionally the amino acids corresponding to 73-86 of SEQ ID NO: 2, or the amino acid corresponding to asparagine 173 of SEQ ID NO: 2, or the amino acids corresponding to 296-314 of SEQ ID NO: 2, if the SFRP is SFRP-1, is/are mutated. Another non-limiting example is a transgenic animal genetically modified such that one or more of a domain selected from the group consisting of the cysteine rich domain (CRD), the netrin domain, and the hyaluronan domain. A test compound may be administered to one or more of these types of animals and the resulting phenotype compared to that of an identical animal to which was administered a placebo and/or an SFRP +/+ animal and/or an SFRP −/− animal. If there is a change in a bone formation parameter (as described above) of the animal administered the test compound as compared to either an identical animal administered a placebo or an SFRP −/− animal, or if there is recovery of an SFRP activity as determined in comparison with an SFRP +/+ animal, then the test compound modulates the Wnt pathway by way of the SFRP protein molecule through the amino acid(s) which was/were mutated.

The present invention furthers our understanding of SFRP/Wnt signaling and bone formation by showing that specific domains and amino acids are important for SFRP/Wnt signaling, specifically the domains corresponding to the cysteine rich domain (CRD), the netrin domain, and the hyaluronan domain and the amino acids corresponding to tyrosine 73 of SEQ ID NO: 2, the amino acids corresponding to lysine 228, 229, 230, 231, 234, 239, 240, 241, 244, and 245 of SEQ ID NO: 2, and additionally the amino acids corresponding to 73-86 of SEQ ID NO: 2, or the amino acid corresponding to asparagine 173 of SEQ ID NO: 2, or the amino acids corresponding to 296- 314 of SEQ ID NO: 2.

### EXAMPLES

### Example 1

### SFRP-1/SFRP-2 Chimeras

### Expression Vectors

hOB SFRP-1 cDNA was cloned as a BamHI-XbaI fragment into the corresponding sites in the pcDNA3 expression plasmid (Invitrogen™, Carlsbad, Calif.). Human SFRP-3 was cloned as an Asp718-XhoI fragment, and human SFRP-2 was cloned as a HindIII-XbaI fragment into pcDNA3 expression plasmids.

The SFRP-1/SFRP-2(1-226)/(214-295) chimera was generated by PCR amplification of the respective fragments and cloning the BamHI-XhoI fragment of hOB SFRP-1 and XhoI-XbaI fragment of human SFRP-2 into the BamHI-XbaI portion of the pcDNA3 expression vector. The following primers were used for the amplification.

| | |
|---|---|
| SFRP-1 5′: | |
| AAAGGATCCGGCATGGGCATCGGGCGCAGC | (SEQ ID NO: 9) |
| SFRP-1 3′: | |
| TATCTCGAGGACAATCTTCTTGTCGCCA, | (SEQ ID NO: 10) |
| SFRP-2 5′: | |
| ATCCTCGAGACCAAGAGCAAGACCA, | (SEQ ID NO: 11) |
| SFRP-2 3′: | |
| TAATCTAGACTAGCACTGCAGCTTGCGGAT, | (SEQ ID NO: 12). |

The SFRP-1/SFRP-2 (1-248/238-295) chimera was isolated by amplifying the HindIII-XbaI fragment of human SFRP-2 (SFRP-2 5′ primer: AAGAAGCTTGTGCTGTGGCTCAAAGACAGC, SEQ ID NO: 13, 3′ primer: TAATCTAGACTAGCACTGCAGCTTGCGGAT, SEQ ID NO: 12), and cloning the HindIII-XbaI fragment into the corresponding sites of the hOB SFRP-1 expression plasmid. The SFRP-1/SFRP-2(1-295/285-295) chimera was generated by creating a unique EcoRI site in hOB SFRP-1 at amino acid 295 (SFRP-1 5′ primer: AAAGGATCCGGCATGGGCATCGGGCGCAGC, SEQ ID NO: 9, and 3′ primer: TTTGAATTCCTTGTTTTTCTTTGTCCCA, SEQ ID NO: 14) and cloning the PCR amplified portion of human SFRP-2 corresponding to amino acids 285-295 (5′ primer: GGGCAGAGAGAATTCAAG, SEQ ID NO: 15, 3′ primer: TAATCTAGACTAGCACTGCAGCTTGCGGAT, SEQ ID NO: 12) into the EcoRI site. The SFRP-2/SFRP-1(1-284/296-314) chimera was generated by cloning the BamHI-EcoRI fragment of human SFRP-2 (PCR 5′ primer: AAAGGATCCGGCATGCTGCAGGGCCCTGGCTCG, SEQ ID NO: 16, 3′ primer: TGCGGGAGATGCGCTTGAATTCTCTCTG, SEQ ID NO: 17) and the EcoRI-XbaI (PCR 5′ primer: TGGGACAAGAAAAACAAGGAATTCA, SEQ ID NO: 18, and 3′ primer: TAATCTAGACTAGCACTGCAGCTTGCGGAT, SEQ ID NO: 12) fragment into the BamHI and XbaI sites of the pcDNA3 expression vector.

All of the plasmid constructs described were verified by DNA sequencing.

### TCF-Luciferase and CMV-βgal Vectors

The TCF-Luciferase vector contained 16 copies of the TCF element upstream of a minimal tk promoter driving the luciferase gene in the pGL3 basic plasmid (Promega, Madison, Wis.), and the CMV β-gal vector was a reporter plasmid from BD Biosciences-Clonetech, Palo Alto, Calif.

### Transfections

The transient transfections were performed using the osteosarcoma cell line, U2OS (ATCC, Rockville, Md.). The U20S cells were maintained in growth medium [McCoy's 5A medium (Invitrogen™) containing 10%(v/v) fetal calf serum (Hyclone, Logan, Utah), 2 mM GlutaMAX™-I (Invitrogen™), and 1× penicillin and streptomycin (Invitrogen™)] and incubated at 37° C. inside a 5% CO₂/95% humidified air incubator. One day prior to transfection, the cells were plated in 96-well tissue culture plates at 2E+4 cells/well in growth medium without antibiotics and incubated at 37° C. inside the 5% CO₂/95% humidified air incubator overnight.

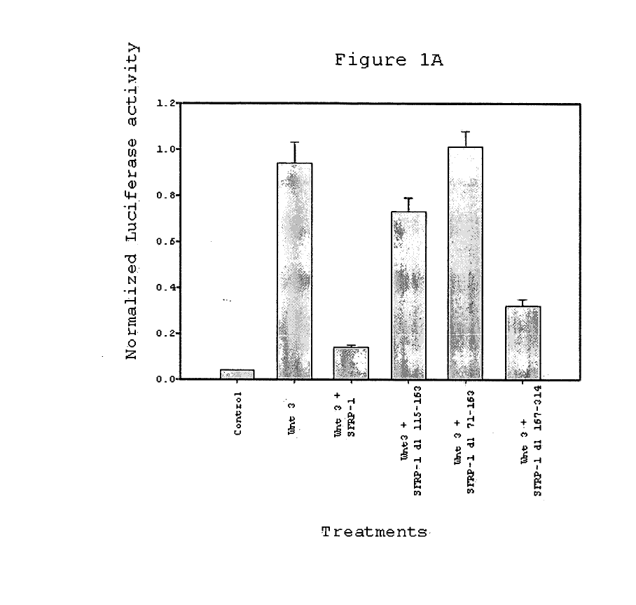

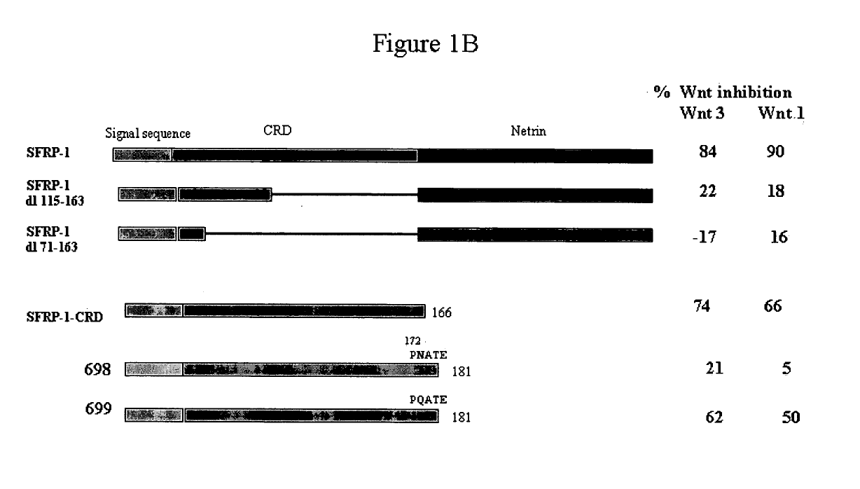

After the overnight incubation, the growth medium was removed, and the cells were washed once with OPTI-MEM® I medium (Invitrogen™; 150 ul per well). The wash medium was removed, and the cells were re-fed with OPTI-MEM® I (100 ul per well). For each transfection, the following plasmid DNA's were diluted together in OPTI-MEM® I medium (25 ul OPTI-MEM® I medium): 16×TCF-luciferase (100 ng per well), Wnt3 or Wnt1 (20ng per well, Upstate Biotechnology, Lake Placid, N.Y.), hOB SFRP-1 (75ng per well), and CMV-βgal (25 ng per well (Clonetech, Palo Alto, Calif.)). Both Wnt1 and Wnt3 were assayed. Unless indicated, the Wnt1 assays gave similar results compared to the Wnt3 assays. Therefore, the data for Wnt1 is only reported in FIG. 1B.

Separately, Lipofectamine™ 2000 reagent (Invitrogen™) was diluted in OPTI-MEM® I medium (0.4 ul Lipofectamine™ 2000 in 25 ul OPTI-MEM® I per well) and incubated at room temperature for 5 minutes. The diluted DNA's were then combined with the diluted Lipofectamine™ 2000, and this mixture was incubated at room temperature for 20 minutes. Fifty microliters of the DNA-Lipofectamine™ 2000 mixture was added to each well, and the plates were incubated at 37° C. in a 5% CO₂/95% humidified air incubator for 4 hours. The medium was then removed, and the cells were washed once with 150 ul per well of phenol red-free RPMI Medium 1640 (Invitrogen™). The wash medium was removed and the cells were re-fed with 100 ul per well of experimental medium [phenol red-free RPMI Medium 1640 containing 2% fetal calf serum, 2 mM GlutaMAX™-1, and 1% penicillin-streptomycin (Invitrogen™)], and the plates were incubated at 37° C. inside a 5% CO₂/95% humidified air incubator overnight.

### Luciferase and β-Gal Assays

After the overnight incubation, the cells were washed twice with 150 ul/well of PBS w/o Ca²⁺ or Mg²⁺ (Invitrogen™) and then lysed with 50 ul/well of 1× cell culture lysis reagent (Promega Corporation, Madison, Wis.) on a shaker at room temperature for 30 minutes. Thirty microliter aliquots of the cell lysates were transferred to 96-well luminometer plates, and luciferase activity was measured in a MicroLumat PLUS luminometer (EG&G Berthold) using 100 ul/well of luciferase substrate (Promega Corporation). Following the injection of substrate, luciferase activity was measured for 10 seconds after a 1.6 second delay. Similarly, 10 ul aliquots of the cell lysates were transferred to separate 96-well luminometer plates, and 50 ul of Galactono chemiluminescent substrate (Tropix®) was added to each well. The plates were covered and incubated on a rotary shaker at room temperature for one hour. β-galactosidase (βgal) activity was measured in a MicroLumat PLUS luminometer using 100 ul/well of Light Emission Accelerator (Tropix®). Following the injection of the accelerator, βgal activity was measured for 10 seconds after a 1.6 second delay. The luciferase and βgal activity data were transferred from the luminometer to a PC and analyzed using the JMP® software (SAS Institute). After the luciferase activity was normalized to βgal, the JMP® program was used to determine the mean and standard deviation for each sample.

### Results and Discussion

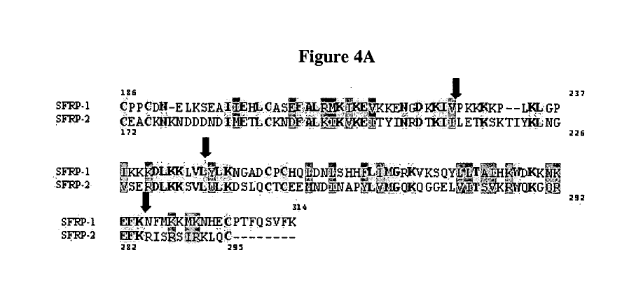

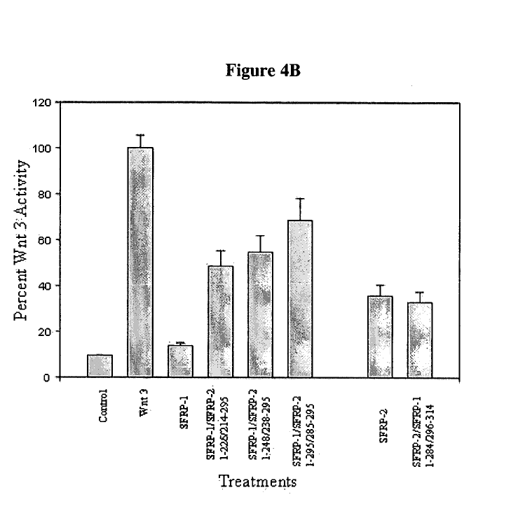

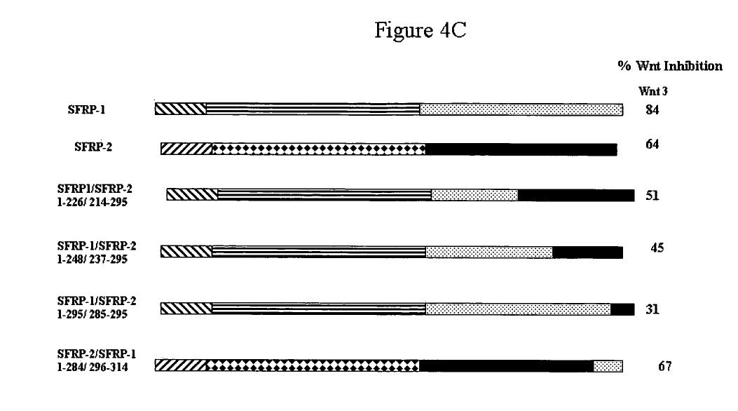

The netrin domain plays a role in the optimal Wnt antagonist function of SFRP-1. In order to address the role of the netrin domain in the Wnt antagonist function of SFRP-1, chimeras of closely related SFRPs such as SFRP-1 and SFRP-2 were isolated and characterized for their Wnt3 antagonist activity. SFRP-2 (SEQ ID NO: 5) exhibits about 36% homology with SFRP-1 (SEQ ID NO: 4) and has pockets of differences within the netrin domain (FIG. 4A). SFRP-2 (SEQ ID NO: 5) is about 25% less active in inhibiting Wnt3-mediated upregulation of the 16×TCF-Luciferase activity.

Three SFRP-1/SFRP-2 chimeras with the netrin domain sequences replaced by the corresponding human SFRP-2 sequences were generated (FIG. 4C) and tested for their Wnt3 antagonist activity in the transfection assay (FIG. 4B). All 3 chimeras exhibited less Wnt 3 antagonist activity compared to SFRP-1. The SFRP-1 chimera with the replacement of the carboxyl-terminal 88 and 66 amino acids with 82 and 58 amino acids of SFRP-2 resulted in about 40% loss of activity. The chimera with the replacement of the carboxyl-terminal 19 amino acids with 11 amino acids of SFRP-2 resulted in the loss of about 70% of the activity. These results clearly indicate that the carboxyl-terminal sequence of SFRP-1 is important for optimal Wnt antagonist function of SFRP-1. The SFRP-2 chimera with replacement of the last 11 amino acids with the corresponding 19 amino acids of SFRP-1 did not affect its Wnt3 antagonist function.

### Example 2

### SFRP-1 CRD

SFRP-1 CRD mutation: Starting from the expression vectors of Example 1, in general, the single point mutations and changes in the small functional domains described were generated by two step PCR using primers containing the desired mutation(s) and amplifying with the 5′ or 3′ primer to generate the PCR fragment with the desired mutation and cloning them into a pcDNA3 vector along with the rest of the hOB SFRP-1 fragment derived either by PCR or by restriction digestion of the hOB SFRP-1 expression plasmid. To mutate the tyrosine to phenylalanine the following primers were used:

SFRP-1 5′ primer: TGCCACAACGTGGGCTTCAAGAAGATG (SEQ ID NO: 19, mutated codon in bold) and

SFRP-1 3′ primer: CCCTCTAGAATCACTTAAACACGGACTGAAAGGTG (SEQ ID NO: 20).

The PCR amplified product was digested with DraIII and XbaI and the purified fragment was cloned into the DraIII-XbaI fragment of hOB SFRP-1 in the pcDNA3 plasmid. Other SFRP-1 tyrosine mutants were generated by replacing the tyrosine codon sequence of the 5′ primer with the desired amino acid codon and cloning the PCR amplified product as indicated above.

SFRP-1 CRD deletion: The CRD deletion mutants were generated by PCR amplification of hOB SFRP-1 with 5′ primers with a BamHil site (AAAGGATCCGGCATGGGCATCGGGCGCAGC, SEQ ID NO: 9) and 3′ primers with an Aat II site (GGGCGCGACGTCCGAGCAGAGGAAGA, SEQ ID NO: 22, CTTCTTGACGTCCACGTTGTGGCACAG, SEQ ID NO: 23) for Δ115-163 and Δ71-163, respectively). The HotStar Taq™ DNA polymerase (Qiagene, Valencia, Calif.) was used for PCR under the following conditions: 94° C. for 15min, denaturation at 940 C for 30sec; annealing at 55° C. for 30sec; extension at 72° C. for 45sec for 5 cycles, 94° C. for 30sec, 58° C. for 30 sec, 72° for 45 sec for 20 cycles, and final extension at 72° C. for 7 min. The purified PCR fragments were digested with BamHI and Aat II, fractionated on an agarose gel, purified and cloned along with the AatII-Xbal fragment from the hOB SFRP-1 plasmid into a BamHI-XbaI fragment of the pcDNA3 expression vector.

All of the plasmid constructs described were verified by DNA sequencing.

Transfections and assays were performed as described in Example 1.

### Results and Discussion

The CRD domain is critical for Wnt antagonist function of SFRP-1 (SEQ ID NO: 2). In U2OS cells, transfection of a Wnt3 expression plasmid along with an optimized 16X TCF-Luciferase reporter led to a 30-fold increase in luciferase activity compared to the reporter alone. Co-transfection of wild-type full length SFRP-1 (SEQ ID NO: 2) led to about 90% inhibition of the Wnt-mediated upregulation of the TCF reporter (FIG. 1A). In order to identify the role of the SFRP-1 CRD in Wnt3 antagonist function, deletion of the CRD was undertaken, and the resulting SFRP-1 mutants (FIG. 1B) were assayed for their Wnt antagonist function (FIG. 1A). A smaller deletion of 48 amino acids (Δ115-163) within the CRD retained about 25% of Wnt3 inhibitory activity. Furthermore, a larger deletion of 92 amino acids (Δ71-163), totally abolished the ability of SFRP-1 to antagonize the Wnt-mediated upregulation of the TCF-reporter. These results indicate that the CRD domain is critical for the Wnt antagonist function of SFRP-1. In support of these data, a truncated SFRP-1 (A167-314) containing the CRD alone is about 80% as active as full length SFRP-1. These results clearly demonstrate that the SFRP-1 CRD is critical in mediating its Wnt antagonist function.

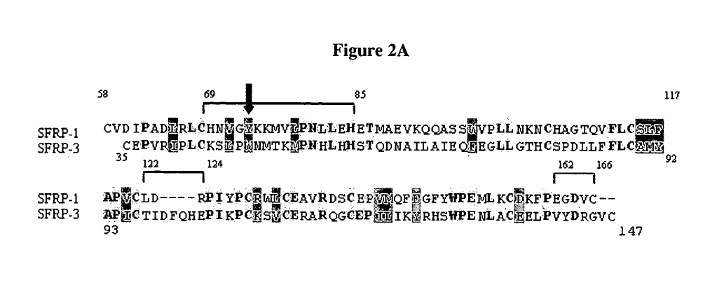

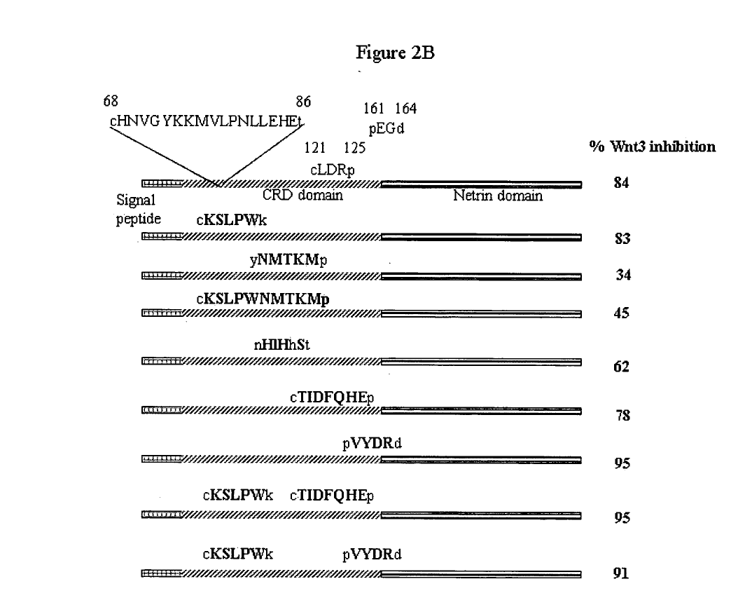

The SFRP CRD contains multiple functional domains required for optimal Wnt antagonist function. The CRD of SFRPs contains 10 conserved cysteine residues, and they form 5 disulphide linkages. Sequence alignment of the CRD domains of SFRP-1 and SFRP-3 indicates about 30% amino acid identity and shows several pockets that differ in their amino acid sequence (FIG. 2A). SFRP-3 is about 60% less active compared to SFRP-1 in its Wnt3 antagonist function, and this difference in the Wnt inhibitory activities could be due to amino acid differences within the CRD of SFRP-3. It is hypothesized that replacement of the SFRP-1 sequence with SFRP-3 would lead to reduced Wnt antagonist activity if the sequence is involved in its function.

Several SFRP-1 mutants with amino acid changes in the CRD that correspond to the SFRP-3 sequence were generated and tested for their Wnt antagonist function (FIG. 2B). The change of amino acids HNVGY (SEQ ID NO: 21) to KSLPW (SEQ ID NO: 30) did not affect the SFRP-1 Wnt antagonist function; however, the change of the SFRP-1 CRD segment from KKMVL (SEQ ID NO: 31) to NMTKM (SEQ ID NO: 32) resulted in a 60% loss of Wnt3 antagonist activity. Combining the two above changes, i.e. HNVGYKKMVL (SEQ ID NO: 33) to KSLPWNMTKM (SEQ ID NO: 34), resulted in a 45% loss of activity. These results clearly indicate that the region between amino acids 74-78 plays an important role in the Wnt antagonist function of SFRP-1. Similarly, a change of amino acids LLEHE (amino acids 81-85; SEQ ID NO: 35) to HLHHS (SEQ ID NO: 36) resulted in about a 30% loss of activity. The results clearly indicate that the 2^{nd} loop of the CRD between cysteine 2 and 3 is critical for its Wnt inhibitory function, and amino acids 74-85 play an important role in the Wnt antagonist function of SFRP-1. Replacement of the SFRP-1 amino acids LDR with TIDEFQHE (SEQ ID NO: 37) residues resulted in a slight decrease in Wnt antagonist activity; however, replacing the amino acids HNVGY (SEQ ID NO: 21) and LDR with KSLPW (SEQ ID NO: 30) and TIDFQHE (SEQ ID NO: 37) resulted in greater Wnt antagonist function. A change of sequence from EG to VYDR (SEQ ID NO: 38) resulted in higher Wnt3 antagonist activity. The above results indicate that there are multiple distinct regions within the CRD domain that are required for optimal Wnt3 antagonist function of SFRP-1.

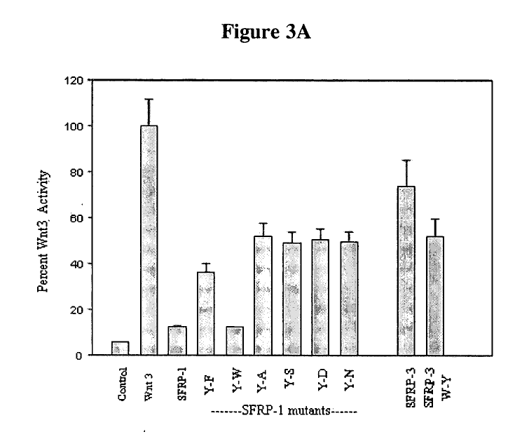

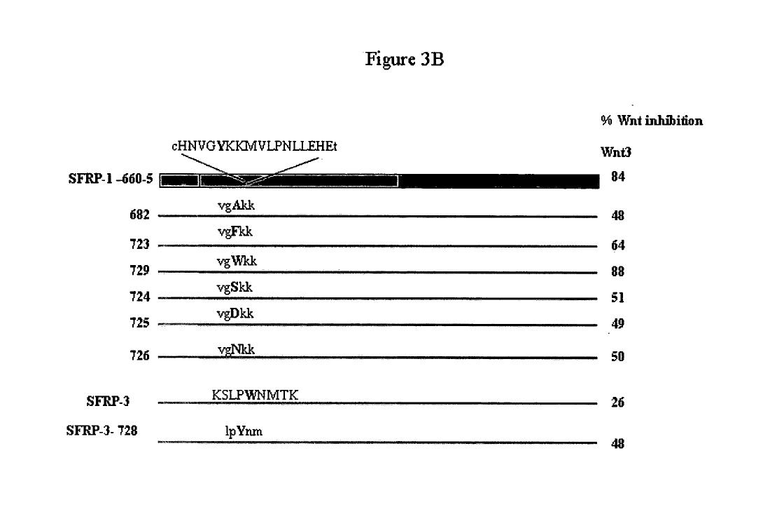

A tyrosine/tryptophan residue in the CRD is critical for Wnt antagonist function of SFRP-1. A tyrosine residue (amino acid 73) within the 2^{nd} loop of the CRD is conserved in SFRP-1 (SEQ ID NOS: 2, 3, 4), −2 (SEQ ID NO: 5), and −5 (SEQ ID NO: 8) and is replaced by tryptophan in SFRP-3 (SEQ ID NO: 6) and SFRP-4 (SEQ ID NO: 7). The tyrosine residue is also conserved in all of the 10 frizzled receptors. In order to determine the role of tyrosine in the Wnt antagonist function of SFRP-1, a number of SFRP-1 mutants were generated (FIG. 3B) and assayed for their ability to antagonize Wnt3-mediated upregulation of the TCF-Luciferase reporter (FIG. 3A). A change of tyrosine to tryptophan in SFRP-1 did not affect its ability to inhibit Wnt3 action; conversely, a change of tryptophan to tyrosine in the CRD of SFRP-3 resulted in increased Wnt3 antagonist activity. The conservative change of the tyrosine residue in SFRP-1 to phenylalanine slightly reduced its Wnt3 antagonist activity. However, the change of tyrosine to alanine, serine, aspartate or asparagine further reduced the Wnt antagonist function to about 40% compared to the wild type SFRP-1. The expression levels of the SFRP-1 mutants were similar to those of the wild type protein, suggesting that the loss of function is not due to the difference in protein levels, but rather is due to the disruption/alteration of the SFRP-1 structure. The functional analysis of the SFRP-1 mutants clearly demonstrates the key role of tyrosine/tryptophan in the overall architecture of SFRP-1 and in its biological function.

### Example 3

### SFRP-1 Hyaluronan Domain

Vectors were generated starting from the expression vectors of Example 1. The hOB SFRP-1 coding sequence contains a unique ApaI site after the hyaluronan-binding region. PCR primers with changes that converted the lysine codons into alanine were obtained and used to generate the SFRP-1 fragment with a mutation of the hyaluronan-binding region. This fragment was cloned along with the ApaI-Xba I fragment of the hOB SFRP-1 into the pcDNA3 expression vector.

The following primers were used:

Set 1.

| | |
|---|---|
| 5′ primer: | |
| AAAGGATCCGGCATGGGCATCGGGCGCAGC | (SEQ ID NO: 9) |
| and | |
| 3′ primer: | |
| GATGGGCCCCAAGGCCAGGGGGGCAGCGGCGGCC | (SEQ ID NO: 39) |
| CCCACAATCTTCTTGTCGCCA. | |

Set 2.

| | |
|---|---|
| 5′ primer: | |
| TTGGGGCCCATCGCCGCTGCCGACCTGGCCGCCC | (SEQ ID NO: 40) |
| TTGTGCTGTACCTGAAG | |
| and | |
| 3′ primer: | |
| CCCTCTAGATTAAACACGGACTGAAAGGT. | (SEQ ID NO: 41) |

The PCR products were digested with Bam HI and ApaI and Set 2 ApaI and XbaI and cloned into the BamHI and XbaI site of the pcDNA3. 1 vector.

All of the plasmid constructs described were verified by DNA sequencing.

Transfections and assays were performed as described in Example 1.

### Results and Discussion

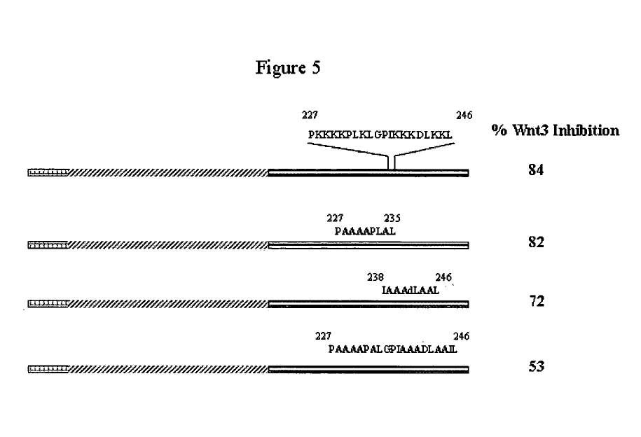

The hyaluronan-binding domain is important for optimal Wnt antagonist function of SFRP-1. The SFRP-1 netrin domain contains clusters of multiple lysine residues referred to as the hyaluronan-binding domain. The effect of the mutation of lysine clusters in SFRP-1 into alanine on Wnt3 antagonist function is shown in FIG. 5. Mutation of the proximal 5 lysine residues into alanine has a minimal effect on the Wnt antagonist function, whereas, the mutation of distal lysine residues resulted in about a 15% loss of activity. The SFRP-1 mutant with a total of 10 lysine residues in the hyaluronan domain changed into alanine resulted in a loss of about 35% of the activity, suggesting that the hyaluronan binding domain is also important for the SFRP-1 Wnt antagonist function.

### Example 4

### SFRP-1 and SFRP-3 Chimera

The expression vectors of Example 1 were used as a starting point. The hOB SFRP-1 coding sequence contains a unique Aat II site (GACGTC, SEQ ID NO: 24 that codes for Asp and Val, respectively) just before the last cysteine residue of the CRD. In human SFRP-3, the corresponding region has the sequence GGCGTG (SEQ ID NO: 25, which codes for Gly and Val, respectively). The sequence in SFRP-3 was converted into an Aat II site by two step PCR using PCR primers containing the desired changes. This resulted in the SFRP-3 coding sequence with a unique Aat II restriction site with a change of the single amino acid Gly to Asp (the resulting sequence termed “mutant SFRP-3”). The BamHI-AatII fragment from hOB SFRP-1 and the Aat II-Xba I fragment from mutant SFRP-3 were cloned into the BamHI-XbaI site of pcDNA3 to generate the SFRP-1/SFRP-3 chimera. Similarly, cloning of the Asp718-Aat II fragment of mutant SFRP3 and the Aat II-Xba I fragment into the Asp718-Xba I site resulted in the SFRP-3/SFRP-1 chimera.

All of the plasmid constructs described were verified by DNA sequencing.

Transfections and assays were performed as described in Example 1.

### Results and Discussion

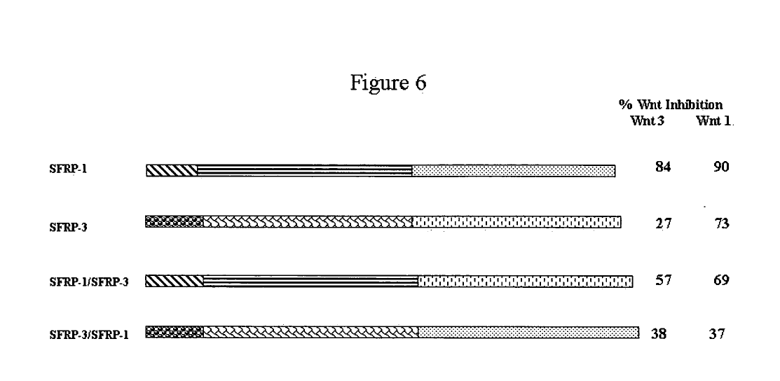

FIG. 6 shows the Wnt inhibition results (for both Wnt3 and Wnt1). SFRP-3/SFRP-1 is more active compared to SFRP-3 in inhibiting the Wnt 3 response. The netrin domain may play an important role to maintain optimal structure for Wnt inhibition. SFRP-3 is more active in inhibiting Wnt 1 compared to Wnt 3. The SFRP-1/SFRP-3 chimera is less active compared to native SFRP-1 in inhibiting the Wnt 3 response.

### Example 5

### Expression Studies with SFRP-1 Mutants

hOB SFRP-1 and mutant plasmids were transfected into U20S (ATCC, Rockville, Md.) or COS (ATCC) cells plated in 60 mm dish using lipofectamine. After 24 hours, the cells were washed with PBS and lysed using RIPA buffer. The clarified cell lysates were fractionated on a 10% NuPAGE gel (Invitrogen™) and the proteins were transferred into nitrocellulose membrane. Western blot was performed following ECL plus detection system (Amersham, London) using SFRP-1 specific polyclonal, H90 (Santa Cruz Biotechnology, Santa Cruz, Calif.), a rabbit antibody that recognizes the C-terminal end of SFRP-1, as a primary antibody and goat anti-rabbit/HRP conjugate as secondary antibody.

A 3′ His tag was generated by PCR amplifying the SFRP-1 with 5′ primer: AAAGGATCCGGCATGGGCATCGGGCGCAGC (SEQ ID NO: 9) and 3′ primer: TTCCTCGAGTCAATGGTGATGGTGATGATGTTTAAACACGGACTGAAAGGTGGGGC A (SEQ ID NO: 42). The PCR amplified product was digested with BamHI and XhoI and was cloned into the corresponding sites in pcDNA3. 1 vector.

The 5′ His tagged hOB SFRP-1 was generated by PCR amplifying SFRP-1 using the following primers.

Product 1:

| | |
|---|---|
| 5′ primer: | |
| AAGGATCCGTCGACCACCATGGGCATCGGGCGC | (SEQ ID NO: 43) |
| and | |
| 3′ primer: | |
| GTACTCGCTAGCCGACCC | (SEQ ID NO: 44) |
| and | |

Product 2: 5′ primer:

TCGGCTAGCCACCACCACCATCACCACGAGTACGACTACGTGAGC (SEQ ID NO: 45) and 3′ primer: AAGAATTCTCTAGAATCACTTAAACACGGA (SEQ ID NO: 46).

The products 1 and 2 were digested with BamHI-NheI and NheI and EcoRI respectively and the purified products were cloned into BamHI and EcoRI site of pcDNA3.1 vector.

### Results and Discussion

Expression studies were performed to ensure expression of the various constructs.

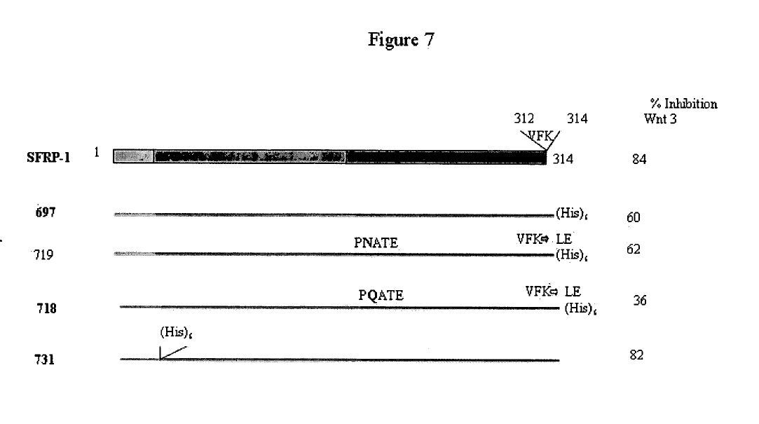

FIG. 7 shows the His tagged proteins and their Wnt3 antagonist activities. Introduction of a His tag at the 3′ end results in loss of about 30% of Wnt inhibitory function of SFRP-1. The SFRP-1 mutant with its last 3 amino acids (VFK) mutated to LE followed by a His tag did not affect its inhibition compared to 3′ His tagged SFRP-1. The 5′ His tagged protein is similar to native SFRP-1 in its Wnt antagonist function. Non-glycosylated protein is much less active, suggesting a role in SFRP-1 function.

Numerous references, including patents, patent applications and various publications, are cited and discussed in the description of this invention. The citation and/or discussion of such references is provided merely to clarify the description of this invention and is not an admission that any such reference is “prior art” to the invention described herein. All references cited and discussed in this specification are incorporated herein by reference in their entirety and to the same extent as if each reference was individually incorporated by reference.

1. A method of screening for a test compound that targets in a secreted frizzled related protein (SFRP) one or more of a domain selected from the group consisting of the cysteine rich domain (CRD), the netrin domain, and the hyaluronan domain, which method comprises:
(i) incubating a first sample comprising a wild-type SFRP and the test compound;
(ii) incubating a second sample comprising a SFRP identical to the SFRP of step (i), except the SFRP has a mutation within or a deletion of one or more of a domain selected from the group consisting of the cysteine rich domain (CRD), the netrin domain, and the hyaluronan domain, and the test compound; and
(ii) determining the amount of test compound bound to SFRP in each sample,
wherein a test compound, that shows increased binding to wild-type SFRP in the first sample compared to the mutant SFRP of the second sample, targets one or more of a domain selected from the group consisting of the cysteine rich domain (CRD), the netrin domain, and the hyaluronan domain in an SFRP.

2. A method for identifying a test compound that targets the Wnt-antagonizing function of a secreted frizzled related protein (SFRP) by way of one or more of a domain selected from the group consisting of the cysteine rich domain (CRD), the netrin domain, and the hyaluronan domain, which method comprises:
(i) incubating a first sample comprising a wild-type SFRP, the test compound and Wnt;
(ii) incubating a second sample comprising a mutant SFRP identical to the SFRP of step (i), except the mutant SFRP has a mutation within or deletion of one or more of a domain selected from the group consisting of the cysteine rich domain (CRD), the netrin domain, and the hyaluronan domain, the test compound and Wnt; and
(iii) determining the Wnt activity in the first and second samples,
wherein an increase in Wnt activity in the second sample compared to the first sample indicates the test compound targets the Wnt-antagonizing function of SFRP by way of one or more of a domain selected from the group consisting of the cysteine rich domain (CRD), the netrin domain, and the hyaluronan domain.

3. A method of screening for a test compound that targets in a secreted frizzled related protein (SFRP) one or more of a domain selected from the group consisting of the cysteine rich domain (CRD), the netrin domain, and the hyaluronan domain, which method comprises:
(i) incubating a first sample comprising a wild-type SFRP, the test compound and Wnt;
(ii) incubating a second sample comprising a mutant SFRP identical to the SFRP of step (i), except the mutant SFRP has a mutation at one or more of a domain selected from the group consisting of the cysteine rich domain (CRD), the netrin domain, and the hyaluronan domain, the test compound and Wnt; and
(ii) determining the amount of SFRP bound to Wnt in each sample,
wherein a test compound, that decreases the binding of wild-type SFRP and Wnt of the first sample compared to the binding of the mutant SFRP and Wnt of the second sample, targets one or more of a domain selected from the group consisting of the cysteine rich domain (CRD), the netrin domain, and the hyaluronan domain in an SFRP.

4. The method of claim 1, 2, or 3, wherein the SFRP is secreted frizzled related protein-1 (SFRP- 1).

5. The method of claim 1, 2, or 3, wherein the SFRP comprises a purification tag at its amino terminus.

6. The method of claim 5, wherein the purification tag is located immediately upstream of the cysteine-rich domain of the SFRP.

7. The method of claim 6, wherein the purification tag is a histidine tag.

8. The method of claim 2 or 3, wherein the Wnt activity is measured by a T-cell factor-luciferase assay.

9. The method of claim 2 or 3, wherein the Wnt is selected from the group consisting of Wnt 3, Wnt 1, Wnt 5, and Wnt 10b.

10. The method of claim 1, 2, or 3, wherein the sample comprises an immortalized human osteoblast cell that expresses a temperature-sensitive mutant of simian virus 40 large T protein antigen, wherein the cell proliferates at about 34° C. but does not proliferate at temperatures exceeding about 37° C. when the T-antigen mutant is inactive.

11. The method of claim 10, wherein the immortalized human osteoblast cell is an hOB-01-C1-PS-09 cell, as deposited with American Type Culture Collection in Manassas, Va. with the designation PTA-785, or progeny thereof.

12. An isolated secreted frizzled related protein (SFRP) in which one or more of a domain selected from the group consisting of the cysteine rich domain (CRD), the netrin domain, and the hyaluronan domain is mutated.

13. An isolated nucleic acid molecule that encodes the SFRP of claim 12.

14. An isolated nucleic acid molecule that is the complement of the nucleic acid molecule of claim 13.

15. An isolated nucleic acid molecule that hybridizes under stringent conditions to the nucleic acid molecule of claim 13 or claim 14.

16. An isolated small molecule identified by the method of claim 1, 2, or 3.

17. A pharmaceutical composition for regulating bone-forming activity in a mammal comprising a small molecule that targets in a secreted frizzled related protein (SFRP) one or more of a domain selected from the group consisting of the cysteine rich domain (CRD), the netrin domain, and the hyaluronan domain, and a pharmaceutically acceptable carrier.

18. The pharmaceutical composition of claim 17, wherein the SFRP is from human osteoblast cells.

19. The pharmaceutical composition of claim 17, wherein the bone-forming activity is the regulation of bone growth.

20. The pharmaceutical composition of claim 17, wherein the bone-forming activity is the regulation of bone density.

21. The pharmaceutical composition of claim 17, further comprising a diluent.

22. The pharmaceutical composition of claim 17, wherein the targeted SFRP is SFRP-1.

23. A pharmaceutical composition for regulating bone-forming activity in a mammal comprising a small molecule that targets in a secreted frizzled related protein-i (SFRP-1) a carboxy terminus of SFRP-1.

24. The pharmaceutical composition of claim 23, wherein the small molecule targets the last 19 amino acids of SFRP-1.

25. A method of modulating Wnt-mediated signaling in a cell comprising contacting the cell with the composition of claim 17, wherein the Wnt activity is regulated.

26. The method of claim 25, wherein the cell is in bone.

27. The method of claim 25, wherein the Wnt is selected from the group consisting of Wnt 3, Wnt 1, Wnt 5, and Wnt 10b.

28. The method of claim 25, wherein the SFRP of the composition is SFRP-1.

29. A method for treating a bone disorder in a mammal comprising the steps of administering the pharmaceutical composition of claim 17.

30. The method of claim 29, wherein the bone disorder is selected from the group consisting of a bone formation disorder, a bone resorption disorder, and a bone density disorder.

31. The method of claim 29, wherein the bone disorder is a degenerative bone disorder.

32. The method of claim 31, wherein the degenerative bone disorder is an osteodegeneration disorder.

33. The method of claim 32, wherein the osteodegeneration disorder is selected from the group consisting of osteopenia, osteoarthritis, and osteoporosis.

34. The method of claim 29, wherein the mammal is a human.

35.-104. (canceled)


SEQUENCE LISTING (56 sequences)

SEQ ID NO: 1 - DNA, 945 bp, Homo sapiens
atgggcatcgggcgcagcgaggggggccgccgcggggcagccctgggcgtgctgctggcgctgggcgcggcgcttctggccgtgggctcggccagcgagtacgactacgtgagcttccagtcggacatcggcccgtaccagagcgggcgcttctacaccaagccacctcagtgcgtggacatccccgcggacctgcggctgtgccacaacgtgggctacaagaagatggtgctgcccaacctgctggagcacgagaccatggcggaggtgaagcagcaggccagcagctgggtgcccctgctcaacaagaactgccacgccggcacccaggtcttcctctgctcgctcttcgcgcccgtctgcctggaccggcccatctacccgtgtcgctggctctgcgaggccgtgcgcgactcgtgcgagccggtcatgcagttcttcggcttctactggcccgagatgcttaagtgtgacaagttccccgagggggacgtctgcatcgccatgacgccgcccaatgccaccgaagcctccaagccccaaggcacaacggtgtgtcctccctgtgacaacgagttgaaatctgaggccatcattgaacatctctgtgccagcgagtttgcactgaggatgaaaataaaagaagtgaaaaaagaaaatggcgacaagaagattgtccccaagaagaagaagcccctgaagttggggcccatcaagaagaaggacctgaagaagcttgtgctgtacctgaagaatggggctgactgtccctgccaccagctggacaacctcagccaccacttcctcatcatgggccgcaaggtgaagagccagtacttgctgacggccatccacaagtgggacaagaaaaacaaggagttcaaaaacttcatgaagaaaatgaaaaaccatgagtgccccacctttcagtccgtgtttaagtga

SEQ ID NO: 2 - protein, 314 aa, Homo sapiens
MGIGRSEGGRRGAALGVLLALGAALLAVGSASEYDYVSFQSDIGPYQSGRFYTKPPQCVDIPADLRLCHNVGYKKMVLPNLLEHETMAEVKQQASSWVPLLNKNCHAGTQVFLCSLFAPVCLDRPIYPCRWLCEAVRDSCEPVMQFFGFYWPEMLKCDKFPEGDVCIAMTPPNATEASKPQGTTVCPPCDNELKSEAIIEHLCASEFALRMKIKEVKKENGDKKIVPKKKKPLKLGPIKKKDLKKLVLYLKNGADCPCHQLDNLSHHFLIMGRKVKSQYLLTAIHKWDKKNKEFKNFMKKMKNHECPTFQSVFK

SEQ ID NO: 3 - protein, 313 aa, Homo sapiens
MGIGRSEGGRRGALGVLLALGAALLAVGSASEYDYVSFQSDIGPYQSGRFYTKPPQCVDIPADLRLCHNVGYKKMVLPNLLEHETMAEVKQQASSWVPLLNKNCHAGTQVFLCSLFAPVCLDRPIYPCRWLCEAVRDSCEPVMQFFGFYWPEMLKCDKFPEGDVCIAMTPPNATEASKPQGTTVCPPCDNELKSEAIIEHLCASEFALRMKIKEVKKENGDKKIVPKKKKPLKLGPIKKKDLKKLVLYLKNGADCPCHQLDNLSHHFLIMGRKVKSQYLLTAIHKWDKKNKEFKNFMKKMKNHECPTFQSVFK

SEQ ID NO: 4 - protein, 314 aa, Homo sapiens
MGIGRSEGGRRGAALGVLLALGAALLAVGSASEYDYVSFQSDIGPYQSGRFYTKPPQCVDIPADLRLCHNVGYKKMVLPNLLEHETMAEVKQQASSWVPLLNKNCHAGTQVFLCSLFAPVCLDRPIYPCRWLCEAVRDSCEPVMQFFGFYWPEMLKCDKFPEGDVCIAMTPPNPTEASKPQGTTVCPPCDNELKSEAIIEHLCASEFALRMKIKEVKKENGDKKIVPKKKKPLKLGPIKKKDLKKLVLYLKNGADCPCHQLDNLSHHFLIMGRKVKSQYLLTAIHKWDKKNKEFKNFMKKMKNHECPTFQSVFK

SEQ ID NO: 5 - protein, 295 aa, Homo sapiens
MLQGPGSLLLLFLASHCCLGSARGLFLFGQPDFSYKRSNCKPIPANLQLCHGIEYQNMRLPNLLGHETMKEVLEQAGAWIPLVMKQCHPDTKKFLCSLFAPVCLDDLDETIQPCHSLCVQVKDRCAPVMSAFGFPWPDMLECDRFPQDNDLCIPLASSDHLLPATEEAPKVCEACKNKNDDDNDIMETLCKNDFALKIKVKEITYINRDTKIILETKSKTIYKLNGVSERDLKKSVLWLKDSLQCTCEEMNDINAPYLVMGQKQGGELVITSVKRWQKGQREFKRISRSIRKLQC

SEQ ID NO: 6 - protein, 325 aa, Homo sapiens [sequence omitted]

SEQ ID NO: 7 - protein, 346 aa, Homo sapiens
MFLSILVALCLWLHLALGVRGAPCEAVRIPMCRHMPWNITRMPNHLHHSTQENAILAIEQYEELVDVNCSAVLRFFLCAMYAPICTLEFLHDPIKPCKSVCQRARDDCEPLMKMYNHSWPESLACDELPVYDRGVCISPEAIVTDLPEDVKWIDITPDMMVQERPLDVDCKRLSPDRCKCKKVKPTLATYLSKNYSYVIHAKIKAVQRSGCNEVTTVVDVKEIFKSSSPIPRTQVPLITNSSCQCPHILPHQDVLIMCYEWRSRMMLLENCLVEKWRDQLSKRSIQWEERLQEQRRTVQDKKKTAGRTSRSNPPKPKGKPPAPKPASPKKNIKTRSAQKRTNPKRV

SEQ ID NO: 8 - protein, 317 aa, Homo sapiens
MRAAAAAGGVRTAALALLLGALHWAPARCEEYDYYGWQAEPLHGRSYSKPPQCLDIPADLPLCHTVGYKRMRLPNLLEHESLAEVKQQASSWLPLLAKRCHSDTQVFLCSLFAPVCLDRPIYPCRSLCEAVRAGCAPLMEAYGFPWPEMLHCHKFPLDNDLCIAVQFGHLPATAPPVTKICAQCEMEHSADGLMEQMCSSDFVVKMRIKEIKIENGDRKLIGAQKKKKLLKPGPLKRKDTKRLVLHMKNGAGCPCPQLDSLAGSFLVMGRKVDGQLLLMAVYRWDKKNKEMKFAVKFMFSYPCSLYYPFFYGAAEPH

SEQ ID NO: 9 - DNA, 30 bp, artificial
  primer
aaaggatccggcatgggcatcgggcgcagc

SEQ ID NO: 10 - DNA, 28 bp, artificial
  primer
tatctcgaggacaatcttcttgtcgcca

SEQ ID NO: 11 - DNA, 25 bp, artificial
  primer
atcctcgagaccaagagcaagacca

SEQ ID NO: 12 - DNA, 30 bp, artificial
  primer
taatctagactagcactgcagcttgcggat

SEQ ID NO: 13 - DNA, 30 bp, artificial
  primer
aagaagcttgtgctgtggctcaaagacagc

SEQ ID NO: 14 - DNA, 28 bp, artificial
  primer
tttgaattccttgtttttctttgtccca

SEQ ID NO: 15 - DNA, 18 bp, artificial
  primer
gggcagagagaattcaag

SEQ ID NO: 16 - DNA, 33 bp, artificial
  primer
aaaggatccggcatgctgcagggccctggctcg

SEQ ID NO: 17 - DNA, 28 bp, artificial
  primer
tgcgggagatgcgcttgaattctctctg

SEQ ID NO: 18 - DNA, 25 bp, artificial
  primer
tgggacaagaaaaacaaggaattca

SEQ ID NO: 19 - DNA, 27 bp, artificial
  primer
tgccacaacgtgggcttcaagaagatg

SEQ ID NO: 20 - DNA, 35 bp, artificial
  primer
ccctctagaatcacttaaacacggactgaaaggtg

SEQ ID NO: 21 - protein, 5 aa, artificial
  internal sequence
HNVGY

SEQ ID NO: 22 - DNA, 26 bp, artificial
  primer
gggcgcgacgtccgagcagaggaaga

SEQ ID NO: 23 - DNA, 27 bp, artificial
  primer
cttcttgacgtccacgttgtggcacag

SEQ ID NO: 24 - DNA, 6 bp, artificial
  restriction site of SEQ ID NO 1
gacgtc

SEQ ID NO: 25 - DNA, 6 bp, artificial
  two codon segment of the nucleic acid sequence of human SFRP-3
ggcgtg

SEQ ID NO: 26 - protein, 388 aa, Homo sapiens
MGLWALLPSWVSTTLLLALTALPAALAANSSGRWWGIVNIASSTNLLTDSKSLQLVLEPSLQLLSRKQRRLIRQNPGILHSVSGGLQSAVRECKWQFRNRRWNCPTAPGPHLFGKIVNRGCRETAFIFAITSAGVTHSVARSCSEGSIESCTCDYRRRGPGGPDWHWGGCSDNIDFGRLFGREFVDSGEKGRDLRFLMNLHNNEAGRTTVFSEMRQECKCHGMSGSCTVRTCWMRLPTLRAVGDVLRDRFDGASRVLYGNRGSNRASRAELLRLEPEDPAHKPPSPHDLVYFEKSPNFCTYSGRLGTAGTAGRACNSSSPALDGCELLCCGRGHRTRTQRVTERCNCTFHWCCHVSCRNCTHTRVLHECLSMAYPYDVPDYASLGPGP

SEQ ID NO: 27 - protein, 373 aa, Homo sapiens
MEPHLLGLLLGLLLGGTRVLAGYPIWWSLALGQQYTSLGSQPLLCGSIPGLVPKQLRFCRNYIEIMPSVAEGVKLGIQECQHQFRGRRWNCTTIDDSLAIFGPVLDKATRESAFVHAIASAGVAFAVTRSCAEGTSTICGCDSHHKGPPGEGWKWGGCSEDADFGVLVSREFADARENRPDARSAMNKHNNEAGRTTILDHMHLKCKCHGLSGSCEVKTCWWAQPDFRAIGDFLKDKYDSASEMVVEKHRESRGWVETLRAKYALFKPPTERDLVYYENSPNFCEPNPETGSFGTRDRTCNVTSHGIDGCDLLCCGRGHNTRTEKRKEKCHCVFHWCCYVSCQECIRIYDVHTCKSMAYPYDVPDYASLGPGP

SEQ ID NO: 28 - DNA, 2602 bp, Homo sapiens
gatctgctggggactgcgccttttgtccccggaggtccctggaagtttgcggcgggacgcgcgcggggaggcggcggaggcagccccgacgtcgcggagaacagggcgcagagccggcatgggcatcgggcgcagcgaggggggccgccgcggggcagccctgggcgtgctgctggcgctgggcgcggcgcttctggccgtgggctcggccagcgagtacgactacgtgagcttccagtcggacatcggcccgtaccagagcgggcgcttctacaccaagccacctcagtgcgtggacatccccgcggacctgcggctgtgccacaacgtgggctacaagaagatggtgctgcccaacctgctggagcacgagaccatggcggaggtgaagcagcaggccagcagctgggtgcccctgctcaacaagaactgccacgccggcacccaggtcttcctctgctcgctcttcgcgcccgtctgcctggaccggcccatctacccgtgtcgctggctctgcgaggccgtgcgcgactcgtgcgagccggtcatgcagttcttcggcttctactggcccgagatgcttaagtgtgacaagttccccgagggggacgtctgcatcgccatgacgccgcccaatgccaccgaagcctccaagccccaaggcacaacggtgtgtcctccctgtgacaacgagttgaaatctgaggccatcattgaacatctctgtgccagcgagtttgcactgaggatgaaaataaaagaagtgaaaaaagaaaatggcgacaagaagattgtccccaagaagaagaagcccctgaagttggggcccatcaagaagaaggacctgaagaagcttgtgctgtacctgaagaatggggctgactgtccctgccaccagctggacaacctcagccaccacttcctcatcatgggccgcaaggtgaagagccagtacttgctgacggccatccacaagtgggacaagaaaaacaaggagttcaaaaacttcatgaagaaaatgaaaaaccatgagtgccccacctttcagtccgtgtttaagtgattctcccgggggcagggaattctgcagatatccagcatggggagggagcctcgggtggggtgggagcgggggggacagtgccccgggaacccggtgggtcacacacacgcactgcgcctgtcagtagtggacattgtaatccagtcggcttgttcttgcagcattcccgctcccttccctccatagccacgctccaaaccccagggtagccgtggccgggtaaagcaagggccatttagattaggaaggtttttaagatccgcaatgtggagcagcagccactgcacaggaggaggtgacaaaccatttccaacagcaacacagccactaaaacacaaaaagggggattgggcggaaagtgagagccagcagcaaaaactacattttgcaacttgttggtgtggatctattggctgatctatgcctttcaactagaaaattctaatgattggcaagtcacgttgttttcaggtccagagtagtttctttctgtctgctttaaatggaaacagactcataccacacttacaattaaggtcaagcccagaaagtgataagtgcagggaggaaaagtgcaagtccattatgtagtagtgacagcaaagggaccaggggagaggcattgccttctctgcccacagtctttccgtgtgattgtctttgaatctgaatcagccagtctcagatgccccaaagtttcggttcctatgagcccggggcatgatctgatccccaagacatgtggaggggcagcctgtgcctgcctttgtgtcagaaaaaggaaaccacagtgagcctgagagagacggcgattttcgggctgagaaggcggtagttttcaaaacacatagttaaaaaagaaacaaatgaaaaaaattttagaacagtccagcaaattgctagtcagggtgaattgtgaaattgggtgaagagcttacgattctaatctcatgttttttccttttcacatttttaaaagaacaatgacaaacacccacttatttttcaaggttttaaaacagtctacattgagcatttgaaaggtgtgctagaacaaggtctcctgatccgtccgaggctgcttcccagaggagcagctctccccaggcatttgccaagggaggcggatttccctggtagtgtagctgtgtggctttccttcctgaagagtccgtggttgccctaaaacctaacaccccctagcaaaactcacagagctttccgtttttttctttcctgtaaagaaacatttcctttgaacttgattgcctatggatcaaagaaattcagaacagcctgcctgtccccccgcactttttacatatatttgtttcatttctgcagatggaaagttgacatgggtggggtgtccccatccagcgagagagtttcaaaagcaaaacatctctgcagtttttcccaagtgccctgagatacttcccaaagcccttatgtttaatcagcgatgtatataagccagttcacttagacaactttacccttcttgtccaatgtacaggaagtagttctaaaaaaaaa

SEQ ID NO: 29 - protein, 7 aa, artificial
  synthetic peptide
  MISC_FEATURE (1)..(1): Xaa is L or V
  MISC_FEATURE (7)..(7): Xaa is L or W
XVDGRWX

SEQ ID NO: 30 - protein, 5 aa, artificial
  internal sequence
KSLPW

SEQ ID NO: 31 - protein, 5 aa, artificial
  internal sequence
KKMVL

SEQ ID NO: 32 - protein, 5 aa, artificial
  internal sequence
NMTKM

SEQ ID NO: 33 - protein, 10 aa, artificial
  internal sequence
HNVGYKKMVL

SEQ ID NO: 34 - protein, 10 aa, artificial
  internal sequence
KSLPWNMTKM

SEQ ID NO: 35 - protein, 5 aa, artificial
  internal sequence
LLEHE

SEQ ID NO: 36 - protein, 5 aa, artificial
  internal sequence
HLHHS

SEQ ID NO: 37 - protein, 8 aa, artificial
  internal sequence
TIDEFQHE

SEQ ID NO: 38 - protein, 4 aa, artificial
  internal sequence
VYDR

SEQ ID NO: 39 - DNA, 55 bp, artificial
  primer
gatgggccccaaggccaggggggcagcggcggcccccacaatcttcttgtcgcca

SEQ ID NO: 40 - DNA, 51 bp, artificial
  primer
ttggggcccatcgccgctgccgacctggccgcccttgtgctgtacctgaag

SEQ ID NO: 41 - DNA, 29 bp, artificial
  primer
ccctctagattaaacacggactgaaaggt

SEQ ID NO: 42 - DNA, 57 bp, artificial
  primer
ttcctcgagtcaatggtgatggtgatgatgtttaaacacggactgaaaggtggggca

SEQ ID NO: 43 - DNA, 33 bp, artificial
  primer
aaggatccgtcgaccaccatgggcatcgggcgc

SEQ ID NO: 44 - DNA, 18 bp, artificial
  primer
gtactcgctagccgaccc

SEQ ID NO: 45 - DNA, 45 bp, artificial
  primer
tcggctagccaccaccaccatcaccacgagtacgactacgtgagc

SEQ ID NO: 46 - DNA, 30 bp, artificial
  primer
aagaattctctagaatcacttaaacacgga

SEQ ID NO: 47 - protein, 38 aa, artificial
  internal sequence
CTDIAYNQTIMPNLLGHTNQEDAGLEVHQFYPLVKVQC

SEQ ID NO: 48 - protein, 38 aa, artificial; identical to SEQ ID NO: 47
  internal sequence

SEQ ID NO: 49 - protein, 38 aa, artificial
  internal sequence
CQDLPYNTTFMPNLLNHYDQQTAALAMEPFHPMVNLDC

SEQ ID NO: 50 - protein, 38 aa, artificial
  internal sequence
CQNLGYNVTKMPNLVGHELQTDAELQLTTFTPLIQYGC

SEQ ID NO: 51 - protein, 38 aa, artificial
  internal sequence
CRGIGYNLTHMPNQFNHDTQDEAGLEVHQFWPLVEIQC

SEQ ID NO: 52 - protein, 38 aa, artificial
  internal sequence
CMKMAYNMTFFPNLMGHYDQSIAAVEMEHFLPLANLEC

SEQ ID NO: 53 - protein, 38 aa, artificial
  internal sequence
CTDIAYNQTILPNLLGHTNQEDAGLEVHQFYPLVKVQC

SEQ ID NO: 54 - protein, 38 aa, artificial
  internal sequence
CKGIGYNYTYMPNQFNHDTQDEAGLEVHQFWPLVEIQC

SEQ ID NO: 55 - protein, 38 aa, artificial
  internal sequence
CRGIGYNLTRMPNLLGHTSQGEAAAELAEFAPLVQYGC

SEQ ID NO: 56 - protein, 38 aa, artificial
  internal sequence
CKDIGYNMTRMPNLMGHENQREAAIQLHEFAPLVEYGC